(19) United States
(12) Patent Application Publication
(10) Pub. No.: US 2022/0227758 A1
(43) Pub. Date: Jul. 21, 2022

(54) IMIDAZOPYRIDINE COMPOUND AS IRAK4 INHIBITOR

(71) Applicant: MEDSHINE DISCOVERY INC., Nanjing, Jiangsu (CN)

(72) Inventors: Jianfei WANG, Shanghai (CN); Jie LI, Shanghai (CN); Haizhong TAN, Shanghai (CN); Yang ZHANG, Shanghai (CN); Jian LI, Shanghai (CN); Shuhui CHEN, Shanghai (CN)

(21) Appl. No.: 17/597,126

(22) PCT Filed: Jun. 24, 2020
(86) PCT No.: PCT/CN2020/098259

§ 371 Date: Dec. 27, 2021

(30) Foreign Application Priority Data
Jun. 26, 2019 (CN) 201910562164.6
Jul. 10, 2019 (CN) 201910619604.7
Dec. 6, 2019 (CN) 201911240851.2
May 28, 2020 (CN) 202010466005.9

(51) Int. Cl. C07D 471/04; A61P 19/02; A61P 35/00

(52) U.S. Cl. CPC C07D 471/04; A61P 35/00; A61P 19/02

(57) ABSTRACT

A class of IRAK4 inhibitors are used in the preparation of drugs for the treatment of diseases related to IRAK4. A compound as represented by formula (II), an isomer thereof or a pharmaceutically acceptable salt thereof is an example of the IRAK4 inhibitors.

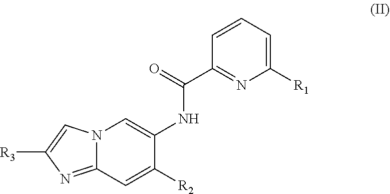

### The present application claims priorities to

CN201910562164.6, filed on Jun. 26, 2019;

CN201910619604.7, filed on Jul. 10, 2019;

CN201911240851.2, filed on Dec. 06, 2019; and

CN202010466005.9, filed on May 28, 2020.

### TECHNICAL FIELD

The invention relates to IRAK4 inhibitors and use thereof in the preparation of a medicament for the treatment of IRAK4-related diseases, and in particular, to a compound of formula (II), an isomer thereof or a pharmaceutically acceptable salt thereof.

### BACKGROUND

Interleukin-1 receptor associated kinase 4 (IRAK4) is a serine/threonine-specific protein kinase, a member of tyrosine-like kinase (TLK) family, and a key node in the innate immune response involving interleukin-1, 18 and 33, and toll-like receptors. After extracellular signal molecules bind to interleukin receptors or toll-like receptors, proteins are recruited to form a MyD88:IRAK4:IRAK1/2 multiprotein complex, leading to IRAK1/2 phosphorylation which mediates a series of downstream signaling. Thus p38, JNK, and NF-κB signaling pathways are activated, eventually promoting the expression of proinflammatory cytokines. Clinical pathology studies have shown that subjects with IRAK4 mutations have resistance against chronic lung disease and inflammatory bowel disease. IRAK4 deficiency is not lethal in itself, and the subjects can survive to adulthood with a reduced risk of infection over age. Therefore, IRAK4 becomes an important therapeutic target attracting extensive research and development interest.

IRAK4-mediated aberrant activation of the TLR/IL-1R pathway has been demonstrated closely associated with the development and progression of several diseases, such as atherosclerosis, rheumatoid arthritis, systemic lupus erythematosus, sepsis, inflammatory bowel disease, asthma, and metabolic syndrome, etc. It has been reported that: in LPS- or CpG-induced PMBCs or THP cells, IRAK4 inhibitors can effectively block the production of a proinflammatory cytokine, tumor necrosis factor TNF-alpha; in a collagen-induced mouse arthritis model, IRAK4 inhibitors can effectively block the generation of TNF-alpha and effectively inhibit the mouse joint swelling; in a mouse OCI-1y10 xenograft tumor model, IRAK4 inhibitors can effectively block the activation of a signaling pathway caused by MyD88-L265P abnormality, and thus, when they are used in combination with BTK inhibitors, PI3K inhibitors and the like, significantly enhance the efficacy of the inhibitors in diffuse large B cell lymphoma DLBCL and promote the apoptosis of tumor cells. Therefore, IRAK4 inhibitors can be widely used for treating various diseases such as inflammatory diseases, immune diseases and tumor diseases, etc. IRAK4 is an important target, and there is remarkable clinical value in developing IRAK4 inhibitors. As shown in the following figure, BAY-1830839 and BAY-1834845 are small molecule IRAK4 inhibitors developed by Bayer, and their clinical studies of treating immune diseases are currently ongoing

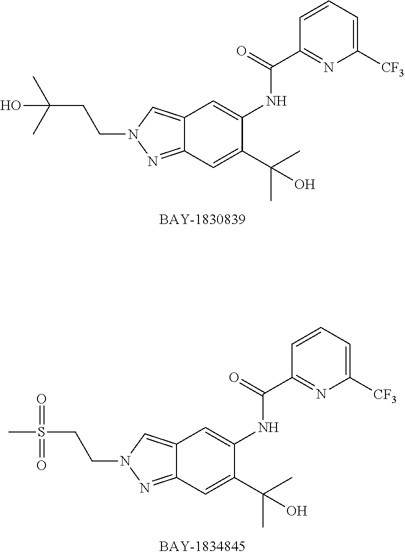 [SMILES: CC(C)(O)CCn1cc2cc(NC(=O)c3cccc(C(F)(F)F)n3)c(C(C)(C)O)cc2n1 ; CC(C)(O)c1cc2nn(CCS(C)(=O)=O)cc2cc1NC(=O)c1cccc(C(F)(F)F)n1]

### SUMMARY

The invention provides a compound of formula (II), an isomer thereof or a pharmaceutically acceptable salt thereof,

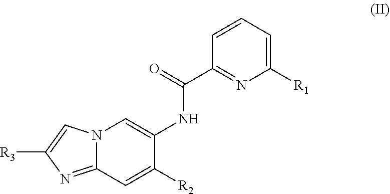

wherein,

R₁ is C₁₋₃ alkyl, wherein the C₁₋₃ alkyl is optionally substituted with 1, 2 or 3 Rₐ;

R₂ is selected from C₁₋₆ alkyl, C₁₋₆ alkoxy, cyclopropyl, azetidinyl,

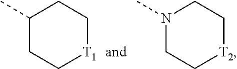

the C₁₋₆ alkyl, C₁₋₆ alkoxy, cyclopropyl, azetidinyl,

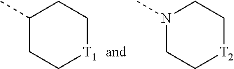

being optionally substituted with 1, 2 or 3 R_{b};

R₃ is C₁₋₆ alkyl, the C₁₋₆ alkyl being optionally substituted with 1, 2 or 3 R_{c};

T₁ is selected from CH₂, NH and O;

T₂ is selected from CH₂, NH and O;

each Rₐ is independently selected from H, F, Cl, Br, I, OH, NH₂, CN and CH₃;

each R_{b} is independently selected from H, F, Cl, Br, I, OH, NH₂, CN, CH₃, —C(═O)—C₁₋₃ alkyl, —C(═O)—C₁₋₃ alkoxy, —C(═O)NH₂ and —COOH, the CH₃, —C(═O)—C₁₋₃ alkyl and —C(═O)—C₁₋₃ alkoxy being optionally substituted with 1, 2 or 3 R;

each R_{c} is independently selected from H, F, Cl, Br, I, OH, NH₂, CN, CH₃, COOH and —S(═O)₂—C₁₋₃ alkyl;

each R is independently selected from H, OH and NH₂.

In some embodiments of the invention, R₁ is CF₃; the other variables are as defined herein.

In some embodiments of the invention, each R_{b} is independently selected from H, F, Cl, OH, NH₂, CN, CH₃, CH₂OH, CH₂NH₂,

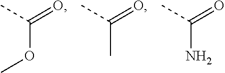

and —COOH; the other variables are as defined herein.

In some embodiments of the invention, R₂ is selected from C₁₋₃ alkyl, C₁₋₃ alkoxy,

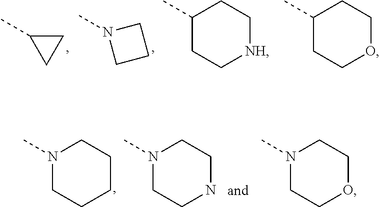

the C₁₋₃ alkyl, C₁₋₃ alkoxy,

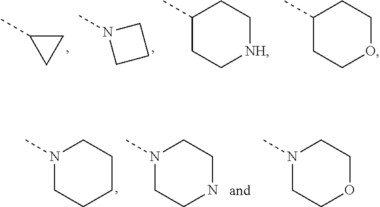

being optionally substituted with 1, 2 or 3 R_{b}; the other variables are as defined herein.

In some embodiments of the invention, R₂ is selected from

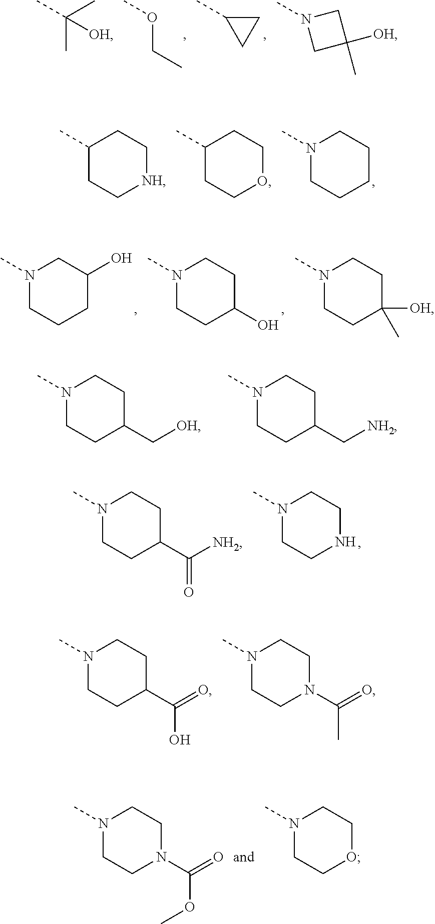

the other variables are as defined herein.

In some embodiments of the invention, each R_{c} is independently selected from H, F, Cl, OH, NH₂, COOH and —S(═O)₂CH₃; the other variables are as defined herein.

In some embodiments of the invention, R₃ is selected from

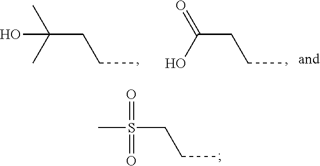

the other variables are as defined herein.

In some embodiments of the invention, the compound, the isomer thereof or the pharmaceutically acceptable salt thereof is selected from the group consisting of:

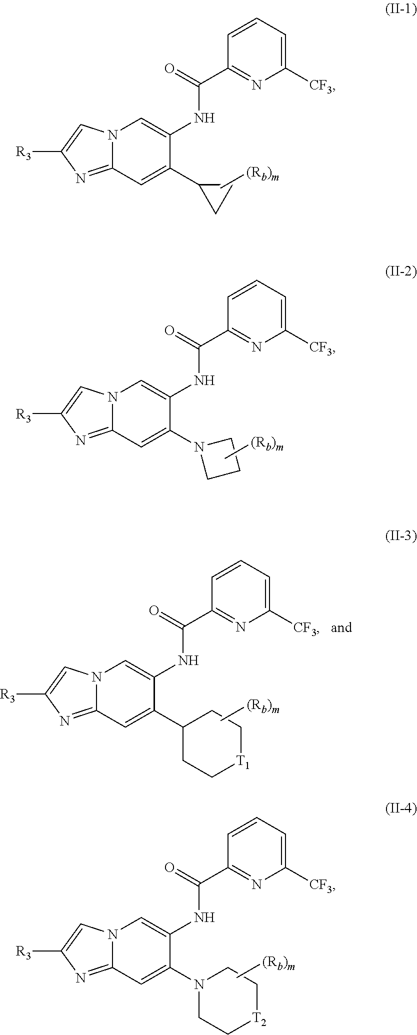

wherein R₃, R_{b}, T₁ and T₂ are as defined herein;

m is selected from 1, 2 and 3.

The invention further provides a compound of formula (I), an isomer or a pharmaceutically acceptable salt thereof,

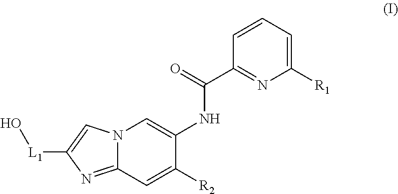

wherein,

R₁ is C₁₋₃ alkyl, wherein the C₁₋₃ alkyl is optionally substituted with 1, 2 or 3 Rₐ;

R₂ is selected from C₃₋₈ cycloalkyl, 3-8 membered heterocycloalkyl, C₁₋₆ alkyl and C₁₋₆ alkoxy, the C₃₋₈ cycloalkyl, 3-8 membered heterocycloalkyl, C₁₋₆ alkyl and C₁₋₆ alkoxy being optionally substituted with 1, 2 or 3 R_{b};

L₁ is selected from C₁₋₆ alkyl, the C₁₋₆ alkyl being optionally substituted with 1, 2 or 3 R_{c};

each Rₐ is independently selected from H, F, Cl, Br, I, OH, NH₂, CN and CH₃;

each R_{b} is independently selected from H, F, Cl, Br, I, OH, NH₂, CN, CH₃, —C(═O)—C₁₋₃ alkyl, —C(═O)—C₁₋₃ alkoxy and —COOH;

each R_{c} is independently selected from H, F, Cl, Br, I, OH, NH₂, CN and CH₃;

the term “hetero-” in the 3-8 membered heterocycloalkyl represents independently being selected from: N, O and NH, the number of the heteroatoms or heteroatom groups being independently selected from 1, 2 and 3.

In some embodiments of the invention, R₁ is CF₃; the other variables are as defined herein.

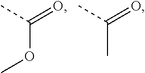

In some embodiments of the invention, R_{b} is selected from H, F, Cl, Br, I, OH, NH₂, CN, CH₃, and —COOH; the other variables are as defined herein.

In some embodiments of the invention, R₂ is selected from piperidinyl, piperazinyl, tetrahydropyrrolyl, tetrahydropyranyl, cyclopropyl, C₁₋₃ alkyl and C₂₋₄ alkoxy, the piperidinyl, piperazinyl, tetrahydropyrrolyl, tetrahydropyranyl, cyclopropyl, C₁₋₃ alkyl and C₂₋₄ alkoxy being optionally substituted with 1, 2 or 3 R_{b}; the other variables are as defined herein.

In some embodiments of the invention, R₂ is selected from

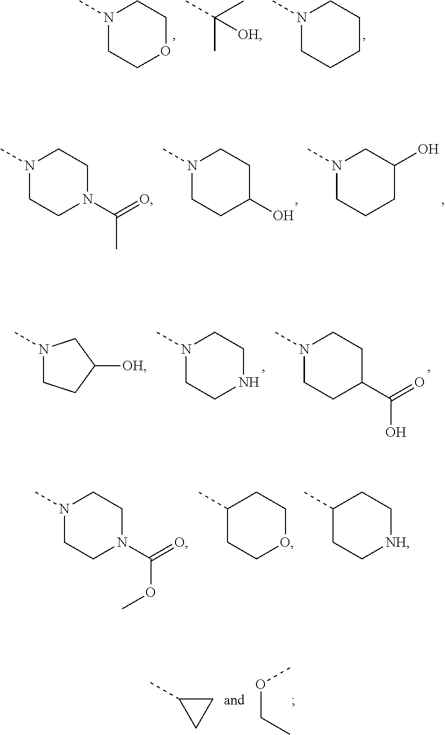

the other variables are as defined herein.

In some embodiments of the invention, L₁ is selected from C₃₋₅ alkyl, the C₃₋₅ alkyl being optionally substituted with 1, 2 or 3 R_{c}; the other variables are as defined herein.

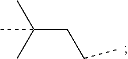

In some embodiments of the invention, L₁ is the other variables are as defined herein.

In some embodiments of the invention, the compound, the isomer thereof or the pharmaceutically acceptable salt thereof is selected from the group consisting of:

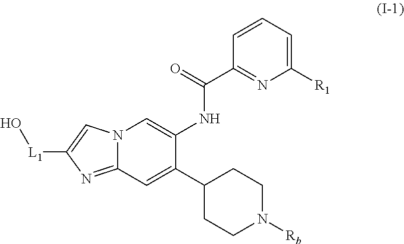

wherein L₁, R₁ and R_{b} are as defined in the present disclosure.

Still some other embodiments of the present disclosure are derived from any combination of the variables described above.

The invention provides compounds of the following formulae, isomers thereof or pharmaceutically acceptable salts thereof:

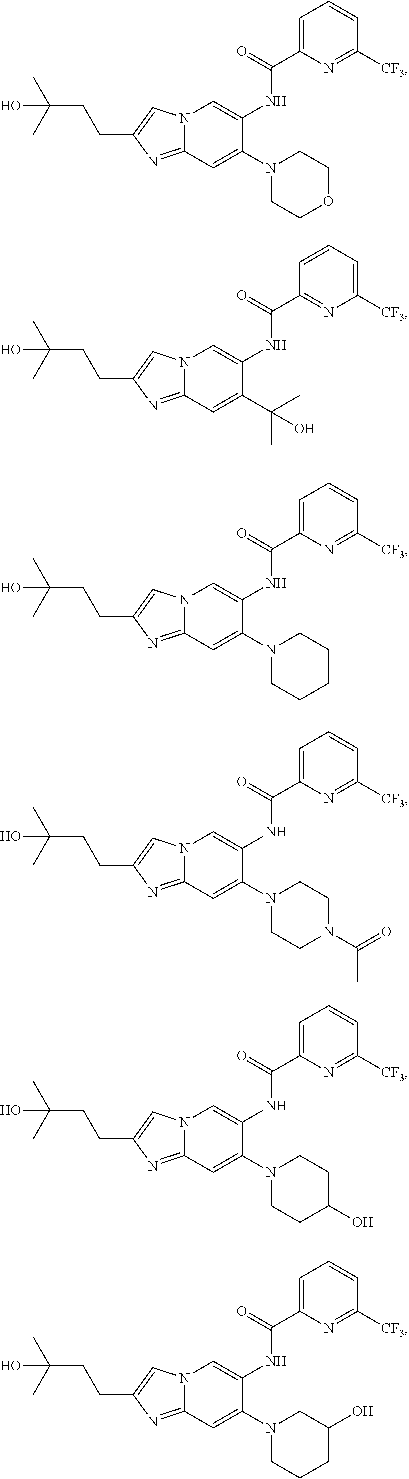

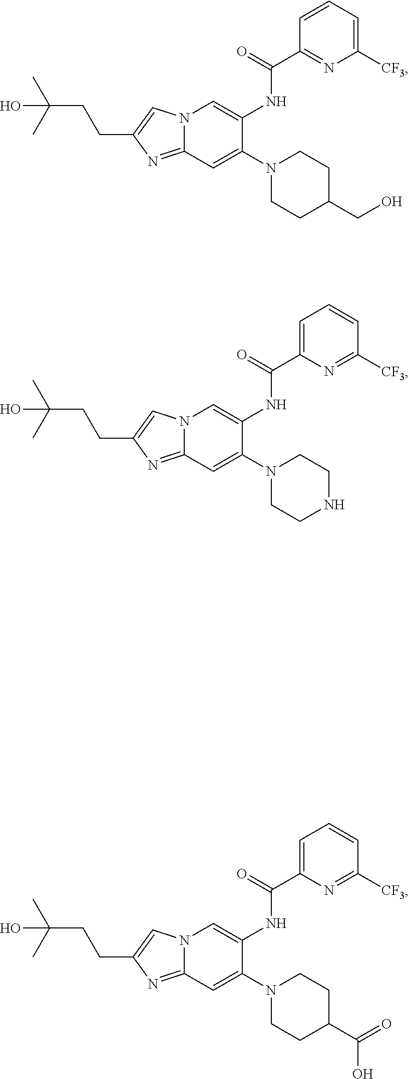 [SMILES: CC(C)(O)CCc1cn2cc(NC(=O)c3cccc(C(F)(F)F)n3)c(N3CCC(CO)CC3)cc2n1 ; CC(C)(O)CCc1cn2cc(NC(=O)c3cccc(C(F)(F)F)n3)c(N3CCNCC3)cc2n1 ; CC(C)(O)CCc1cn2cc(NC(=O)c3cccc(C(F)(F)F)n3)c(N3CCC(C(=O)O)CC3)cc2n1]

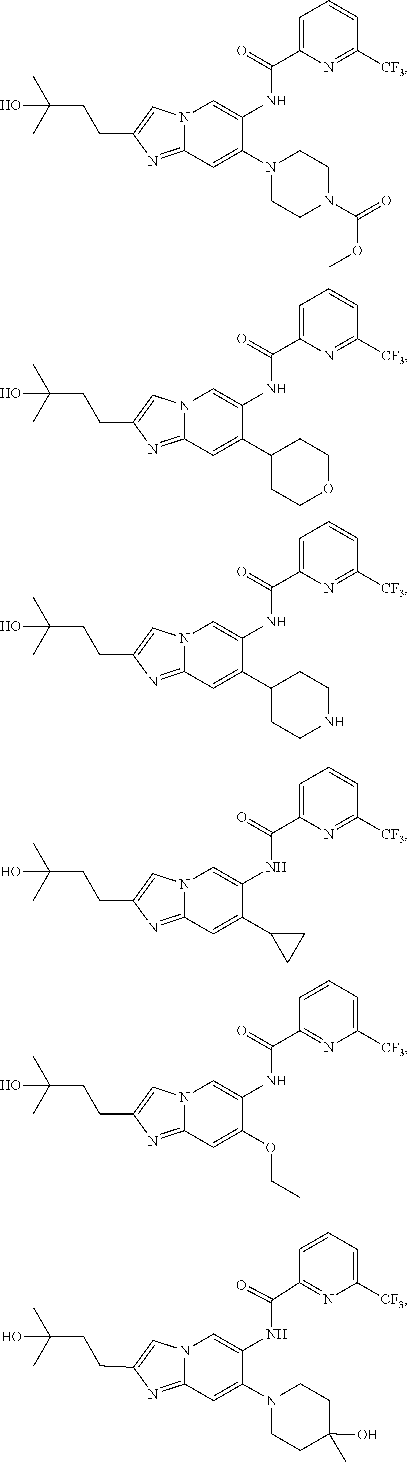

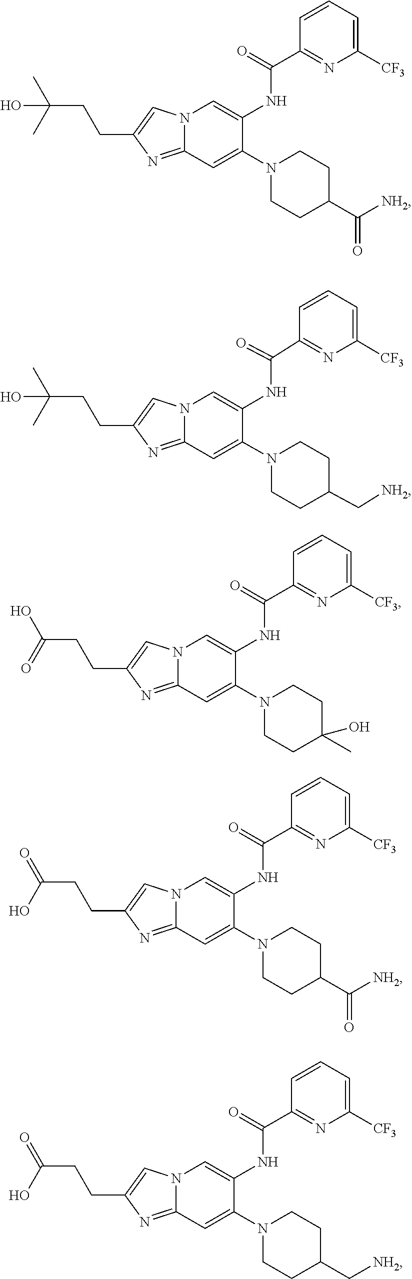

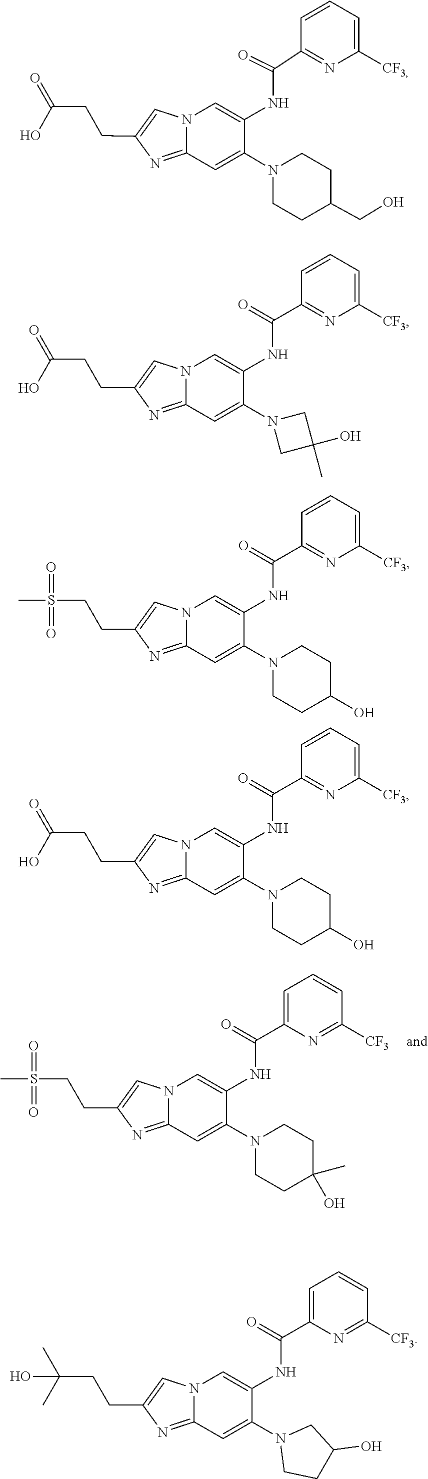

In some embodiments of the invention, the compounds, the isomers thereof or the pharmaceutically acceptable salts thereof are selected from:

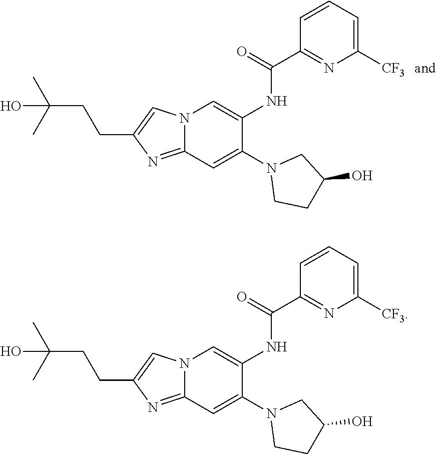

The invention also provides a pharmaceutical composition comprising a therapeutically effective amount of the compound, the isomer thereof or the pharmaceutically acceptable salt thereof as an active ingredient, and a pharmaceutically acceptable carrier.

The invention also provides use of the compound, the isomer thereof or the pharmaceutically acceptable salt thereof, or the pharmaceutical composition thereof in the preparation of a medicament for the treatment of an IRAK4-related disease.

### Technical Effects

The compounds of the invention generally exhibit relatively good inhibitory activity against IRAK4. The compounds of the invention generally showed superior activity for inhibiting TNF-alpha generation in cells in a THP-1 cell activity assay, and good anti-inflammatory effect in a collagen-induced mouse arthritis model.

### Definitions and Description

Unless otherwise stated, the following terms and phrases used herein are intended to have the following meanings. A particular term or phrase, unless otherwise specifically defined, should not be considered as uncertain or unclear, but shall be construed as its common meaning. When referring to a trade name, it is intended to refer to its corresponding commercial product or its active ingredient. The term “pharmaceutically acceptable” is used herein for those compounds, materials, compositions, and/or dosage forms which are, within the scope of sound medical judgment, suitable for use in contact with the tissues of human beings and animals without excessive toxicity, irritation, allergic response, or other problems or complications, and commensurate with a reasonable benefit/risk ratio.

The term “pharmaceutically acceptable salt” refers to a salt of the compound of the invention, which is prepared from the compound having particular substituents disclosed according to the invention and a relatively nontoxic acid or base. When the compound of the invention contains a relatively acidic functional group, a base addition salt can be obtained by contacting such a compound with a sufficient amount of a base in a pure solution or a suitable inert solvent. Pharmaceutically acceptable base addition salts include sodium, potassium, calcium, ammonium, organic amine, or magnesium salts, or similar salts. When the compound of the invention contains a relatively basic functional group, an acid addition salt can be obtained by contacting such a compound with a sufficient amount of an acid in a pure solution or a suitable inert solvent. Examples of pharmaceutically acceptable acid addition salts include salts derived from inorganic acids, such as hydrochloric acid, hydrobromic acid, nitric acid, carbonic acid, bicarbonate, phosphoric acid, monohydrogen phosphate, dihydrogen phosphate, sulfuric acid, hydrogen sulfate, hydroiodic acid and phosphorous acid, etc; and salts derived from organic acids, such as acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, tartaric acid and methanesulfonic acid, etc.

Also included are salts of amino acids (e.g., arginine, etc) and salts of organic acids such as glucuronic acid, etc. Certain specific compounds of the invention contain both basic and acidic functional groups that allow the compounds to be converted into either base or acid addition salts.

The pharmaceutically acceptable salts of the invention can be synthesized from a parent compound having an acidic or basic group by conventional chemical methods. In general, such salts are prepared by the following method: reacting the free acid or base form of the compound with a stoichiometric amount of the appropriate base or acid in water or an organic solvent or a mixture thereof.

The compound of the invention may be present as a specific geometric isomer or stereoisomer. All such compounds are contemplated herein, including cis- and trans-isomers, (−)- and (+)-enantiomers, (R)- and (S)-enantiomers, diastereoisomers, (D)-isomers, (L)-isomers, and racemic mixtures and other mixtures thereof, such as an enantiomer or diastereomer enriched mixture, all of which are encompassed within the scope of the invention. Substituents such as alkyl may have an additional asymmetric carbon atom. All these isomers and mixtures thereof are encompassed within the scope of the invention.

Unless otherwise stated, the term “enantiomer” or “optical isomer” refers to stereoisomers that are mirror images of each other.

Unless otherwise stated, the term “cis-trans isomer” or “geometric isomer” results from the inability of a single bond of a ring carbon atom or a double bond to rotate freely.

Unless otherwise stated, the term “diastereoisomer” refers to stereoisomers in which molecules each have two or more chiral centers and are not mirror images of each other.

Unless otherwise stated, “(+)” stands for dextrorotation, “(−)” stands for levorotation, and “(±)” stands for racemization.

Unless otherwise stated, the absolute configuration of a stereogenic center is represented by a wedged solid bond ( [symbol] ) and a wedged dashed bond ( [symbol] ), and the relative configuration of a stereogenic center is represented by a straight solid bond ( [symbol] ) and a straight dashed bond ( [symbol] ). A wavy line ( [symbol] ) represents a wedged solid bond ( [symbol] ) or a wedged dashed bond ( [symbol] ) or a wavy line ( [symbol] ) represents a straight solid bond ( [symbol] ) and a straight dashed bond ( [symbol] ).

Unless otherwise stated, when a double bond structure such as a carbon-carbon double bond, a carbon-nitrogen double bond, and a nitrogen-nitrogen double bond is present in the compound, and each atom on the double bond is linked to two different substituents (in the double bond including an nitrogen atom, a lone pair of electrons on the nitrogen atom is regarded as a substituent to which the nitrogen atom is linked), if the atom on the double bond of the compound and its substituents are linked using a wavy line ( [symbol] ) it means that the compound exists in the form of a (Z)-type isomer, a (E)-type isomer, or a mixture of the two isomers. For example, the following formula (A) represents that the compound exists in the form of a single isomer of formula (A-1) or formula (A-2) or in the form of a mixture of both isomers of formula (A-1) and formula (A-2); the following formula (B) represents that the compound exists in the form of a single isomer of formula (B-1) or formula (B-2) or in the form of a mixture of both isomers of formula (B-1) and formula (B-2); and the following formula (C) represents that the compound exists in the form of a single isomer of formula (C-1) or formula (C-2) or in the form of a mixture of both isomers of formula (C-1) and formula (C-2).

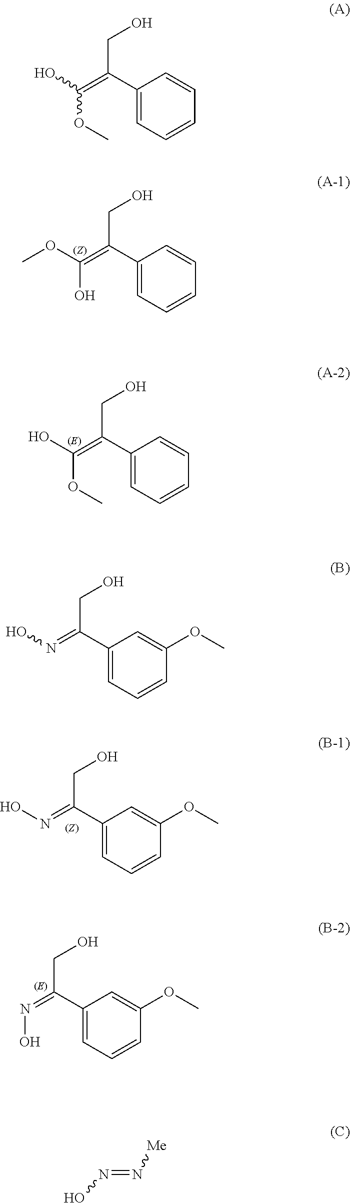

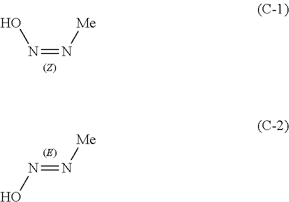

Unless otherwise stated, the term “tautomer” or “tautomeric form” means that isomers with different functional groups are in dynamic equilibrium at room temperature and can be rapidly converted into each other. If tautomer is possible (e.g., in solution), the chemical equilibrium of the tautomers can be achieved. For example, a proton tautomer, also known as a prototropic tautomer, includes interconversion by proton migration, such as keto-enol isomerism and imine-enamine isomerism. A valence isomer includes interconversion by recombination of some bonding electrons. A specific example of the keto-enol tautomerism is the interconversion between the tautomers pentane-2,4-dione and 4-hydroxypent-3-en-2-one.

Unless otherwise stated, the term “enriched with one isomer”, “isomer enriched”, “enriched with one enantiomer” or “enantiomer enriched” means that the content of one of the isomers or enantiomers is less than 100% and more than or equal to 60%, 70%, 80%, 90%, 95%, 96%, 97%, 98%, 99%, 99.5%, 99.6%, 99.7%, 99.8% or 99.9%.

Unless otherwise stated, the term “isomeric excess” or “enantiomeric excess” refers to the difference between the relative percentages of two isomers or enantiomers. For example, if the content of one isomer or enantiomer is 90% and the content of the other isomer or enantiomer is 10%, the isomeric or enantiomeric excess (ee) is 80%.

Optically active (R)- and (S)-isomers and D and L isomers can be prepared by chiral synthesis or chiral reagents or other conventional techniques. If one enantiomer of a certain compound disclosed herein is to be obtained, the desired pure enantiomer can be prepared by asymmetric synthesis or derivatization using a chiral auxiliary, wherein the resulting diastereoisomeric mixture is separated and the auxiliary group is cleaved. Alternatively, when the molecule contains a basic functional group (such as amino) or an acidic functional group (such as carboxyl), the compound reacts with an appropriate optically active acid or base to form a salt of the diastereoisomer, which is then subjected to resolution of diastereoisomer through conventional methods in the art to acquire the pure enantiomer. Furthermore, the enantiomer and the diastereoisomer are generally isolated through chromatography using a chiral stationary phase, optionally in combination with chemical derivatization (e.g., carbamate generated from amines).

The compounds of the invention may contain an unnatural proportion of atomic isotope at one or more of the atoms that constitute the compound. For example, the compound may be labeled with a radioisotope, such as tritium (³H), iodine-125 (¹²⁵I), or C-14 (¹⁴C). For another example, hydrogen can be substituted by deuterium to form a deuterated drug, and the bond formed by deuterium and carbon is firmer than that formed by common hydrogen and carbon. Compared with an un-deuterated drug, the deuterated drug has the advantages of reduced toxic side effect, increased stability, enhanced efficacy, prolonged biological half-life and the like. All isotopic variations of the compound of the invention, whether radioactive or not, are encompassed within the scope of the present disclosure. “Optional” or “optionally” means that the subsequently described event or circumstance may, but does not necessarily, occur, and the description includes instances where the event or circumstance occurs and instances where it does not.

The term “substituted” means that one or more hydrogen atoms on a specific atom are substituted by substituents which may include deuterium and hydrogen variants, as long as the valence of the specific atom is normal and the substituted compound is stable. When the substituent is an oxygen (i.e., ═O), it means that two hydrogen atoms are substituted. Substitution with oxygen does not occur on aromatic groups. The term “optionally substituted” means that an atom can be substituted with a substituent or not. Unless otherwise specified, the type and number of the substituent may be arbitrary as long as being chemically achievable.

When any variable (e.g., R) occurs more than once in the constitution or structure of a compound, the variable is independently defined in each case. Thus, for example, if a group is substituted with 0-2 R, the group can be optionally substituted with up to two R, and the definition of R in each case is independent. Furthermore, a combination of a substituent and/or a variant thereof is permissible only if the combination can result in a stable compound.

When the number of a linking group is 0, for example, —(CRR)₀—, it means that the linking group is a single bond.

When one of variables is selected from single bond, the two groups bonding by this variable are bonded directly. For example, in A—L—Z, when L represents a single bond, it means that the structure is actually A-Z.

When a substituent is absent, it means that the substituent does not exist. For example, when X in A-X is absent, the structure is actually A. When it is not specified by which atom the listed substituent is connected to the group to be substituted, the substituent can be connected via any atom of the group. For example, pyridinyl as a substituent can be connected to the group to be substituted via any carbon atom on the pyridine ring.

When the enumerative linking group does not indicate the direction for linking, the direction for linking is arbitrary. For example, when the linking group L contained in

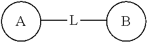

is —M—W—, —M—W— can either link ring A and ring B in a direction same as left-to-right reading order to form

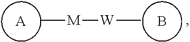

or link ring A and ring B in an opposing direction to form

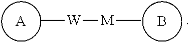

A combination of the linking group, a sub stituent and/or a variant thereof is permissible only if the combination can result in a stable compound.

Unless otherwise specified, when a group has one or more connectable sites, any one or more of the sites of the group may be connected to other groups by chemical bonds. When there is no designated connecting mode for a chemical bond and H atoms are present at a connectable site, the number of the H atoms at the connectable site is correspondingly reduced based on the number of the connected chemical bonds, and a group with a corresponding valence number is thus formed. The chemical bond that connects the site to another group may be represented by a straight solid bond ( [symbol] ) a straight dashed line bond ( [symbol] ), or a wavy line (

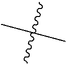

). For example, the straight solid bond in —OCH₃ refers to being connected to another group via the oxygen atom in the group; the straight dashed bond in

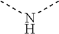

refers to being connected to another group via two ends of the nitrogen atom in the group; the wavy line in

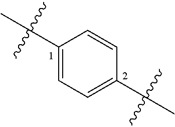 [SMILES: *c1ccc(*)cc1]

refers to being connected to another group via the carbon atoms at positions 1 and 2 in the phenyl group;

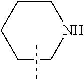

means that any connectable site on the piperidinyl can be connected to another group via 1 bond, and at least 4 connecting modes

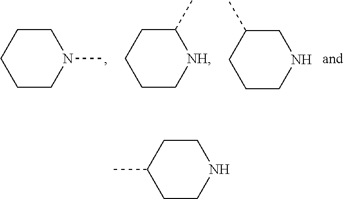

are possible; even if —N— is connected to an H atom,

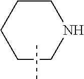

includes the connecting mode of

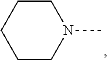

except that when 1 bond is connected to a site, the number of H at that site is correspondingly reduced by 1 and a monovalent piperidinyl is thus formed. Unless otherwise specified, the term “C₁₋₆ alkyl” refers to a linear or branched saturated hydrocarbon group consisting of 1 to 6 carbon atoms. The C₁₋₆ alkyl includes C₁₋₅, C₁₋₄, C₁₋₃, C₁₋₂, C₂₋₆, C₂₋₄, C₆, and C₅ alkyl, etc., and may be monovalent (e.g., methyl), divalent (e.g., methylene), or polyvalent (e.g., methine group). Examples of C₁₋₆ alkyl include, but are not limited to, methyl (Me), ethyl (Et), propyl (including n-propyl and isopropyl), butyl (including n-butyl, isobutyl, s-butyl, and t-butyl), pentyl (including n-pentyl, isopentyl, and neopentyl), and hexyl etc.

Unless otherwise specified, the term “C₁₋₃ alkyl” refers to a linear or branched saturated hydrocarbon group consisting of 1 to 3 carbon atoms. The C₁₋₃ alkyl includes C₁₋₂, C₂₋₃ alkyl, etc., and may be monovalent (e.g., methyl), divalent (e.g., methylene), or polyvalent (e.g., methine group). Examples of C₁₋₃ alkyl include, but are not limited to, methyl (Me), ethyl (Et), and propyl (including n-propyl and isopropyl), etc.

Unless otherwise specified, the term “C₃₋₅ alkyl” refers to a linear or branched saturated hydrocarbon group consisting of 3 to 5 carbon atoms. The C₃₋₅ alkyl includes C₃₋₄, C₅ alkyl, etc., and may be monovalent, divalent or polyvalent. Examples of C₃₋₅ alkyl include, but are not limited to, propyl (including n-propyl and isopropyl), butyl (including n-butyl, isobutyl, s-butyl, and t-butyl), and pentyl (including n-pentyl, isopentyl, and neopentyl), etc.

Unless otherwise specified, the term “C₁₋₆ alkoxy” refers to those alkyl groups that each contains 1 to 6 carbon atoms and is connected to the rest part of the molecule through an oxygen atom. The C₁₋₆ alkoxy includes C_{1- 4,} C_{1- 3,} C₁₋₂, C₂₋₆, C₂₋₄, C₆, C₅, C₄ and C₃ alkoxy, etc. Examples of C₁₋₆ alkoxy include, but are not limited to, methoxy, ethoxy, propoxy (including n-propoxy and isopropoxy), butoxy (including n-butoxy, isobutoxy, s-butoxy and t-butoxy), pentoxy (including n-pentoxy, isopentoxy and neopentoxy), and hexyloxy, etc.

Unless otherwise specified, the term “C₁₋₃ alkoxy” refers to those alkyl groups that each contains 1 to 3 carbon atoms and is connected to the rest part of the molecule through an oxygen atom. The C₁₋₃ alkoxy includes C₁₋₂, C₂₋₃, C₃ and C₂ alkoxy, etc. Examples of C₁₋₃ alkoxy include, but are not limited to, methoxy, ethoxy, and propoxy (including n-propoxy and isopropoxy), etc.

Unless otherwise specified, “C₃₋₈ cycloalkyl” refers to a saturated cyclic hydrocarbon group consisting of 3 to 8 carbon atoms. This includes monocyclic and bicyclic systems, wherein the bicyclic system includes spirocyclic, fused and bridged rings. The C₃₋₈ cycloalkyl includes C₃₋₆, C₃₋₅, C₄₋₈, C₄₋₆, C₄₋₅, C₅₋₈, C₅₋₆ cycloalkyl, or the like, and may be monovalent, divalent, or polyvalent. Examples of C₃₋₈ cycloalkyl include, but are not limited to, cyclopropyl, cyclobutyl, cyclopentyl, cyclohexyl, cycloheptyl, norbornyl, and [2.2.2]bicyclooctane, etc.

Unless otherwise specified, the term “3-8 membered heterocycloalkyl”, by itself or in combination with other terms, refers to a saturated cyclic group consisting of 3 to 8 ring atoms, of which 1, 2, 3, or 4 ring atoms are heteroatoms independently selected from the group consisting of O, S and N, with the remaining being carbon atoms. The nitrogen atom is optionally quaternized, and the nitrogen and sulfur heteroatoms can be optionally oxidized (i.e., NO and S(O)ₚ, where p is 1 or 2). This includes monocyclic and bicyclic systems, wherein the bicyclic system includes spirocyclic, fused, and bridged rings. Furthermore, with respect to the “3-8 membered heterocycloalkyl”, a heteroatom may occupy the position where the heterocycloalkyl is connected to the rest of the molecule. The 3-8 membered heterocycloalkyl includes 3-6 membered, 3-5 membered, 4-6 membered, 5-6 membered, 4 membered, 5 membered and 6 membered heterocycloalkyl, etc. Examples of 3-8 membered heterocycloalkyl include, but are not limited to, azetidinyl, oxetanyl, thietanyl, pyrrolidinyl, pyrazolidinyl, imidazolidinyl, tetrahydrothienyl (including tetrahydrothien-2-yl, tetrahydrothien-3 -yl, etc.), tetrahydrofuranyl (including tetrahydrofuran-2-yl, etc.), tetrahydropyranyl, piperidinyl (including 1-piperidinyl, 2-piperidinyl, 3-piperidinyl, etc.), piperazinyl (including 1-piperazinyl, 2-piperazinyl, etc.), morpholinyl (including 3-morpholinyl, 4-morpholinyl, etc.), dioxanyl, dithianyl, isoxazolidinyl, isothiazolidinyl, 1,2-oxazinyl, 1,2-thiazinyl, hexahydropyridazinyl, homopiperazinyl, homopiperidinyl, and dioxepanyl, etc.

The term “leaving group” refers to a functional group or atom that can be replaced by another functional group or atom through a substitution reaction (e.g., nucleophilic substitution). For example, representative leaving groups include triflate; chlorine, bromine and iodine; sulfonate groups, such as mesylate, tosylate, p-bromobenzenesulfonate and p-toluenesulfonate, etc; acyloxy groups, such as acetoxy, and trifluoroacetoxy, etc.

The term “protective group” includes, but is not limited to, “amino protective group”, “hydroxy protective group” or “sulfydryl protective group”. The term “amino protective group” refers to a protective group suitable for preventing side reactions at the nitrogen atom of the amino. Representative amino protective groups include, but are not limited to: formyl; acyl, such as alkanoyl (such as acetyl, trichloroacetyl or trifluoroacetyl); alkoxycarbonyl, such as t-butoxycarbonyl (Boc);

arylmethyloxycarbonyl, such as benzyloxycarbonyl (Cbz) and 9-fluorenylmethyloxycarbonyl (Fmoc); arylmethyl, such as benzyl (Bn), trityl (Tr), 1,1-di-(4′-methoxyphenyl)methyl; and silyl, such as trimethylsilyl (TMS) and t-butyldimethylsilyl (TBS), etc. The term “hydroxy protective group” refers to a protective group suitable for preventing side reactions of the hydroxy group. Representative hydroxy protective groups include, but are not limited to: alkyl, such as methyl, ethyl, and t-butyl; acyl, such as alkanoyl (such as acetyl); arylmethyl, such as benzyl (Bn), p-methoxybenzyl (PMB), 9-fluorenylmethyl (Fm) and diphenylmethyl (DPM); and silyl, such as trimethylsilyl (TMS) and t-butyldimethylsilyl (TBS), etc.

The compounds of the invention can be prepared by a variety of synthetic methods well known to those skilled in the art, including the specific examples listed below, examples formed by combinations thereof with other chemical synthetic methods, and equivalents thereof known to those skilled in the art. Preferred examples include, but are not limited to, the examples of the present disclosure. The structures of the compounds of the invention can be confirmed by conventional methods well known to those skilled in the art, and if the invention relates to an absolute configuration of the compound, the absolute configuration can be confirmed by means of conventional techniques in the art. For example, by a single crystal X-ray diffraction (SXRD), diffraction intensity data of the obtained single crystal are collected using a Bruker D8 venture diffractometer in the CuKα radiation, by φ/w scanning. After the data collection, the crystal structure is further analyzed using a direct method (Shelxs97), so as to confirm the absolute configuration.

The solvent used in the invention can be commercially available. The invention employs the following abbreviations: ACN represents acetonitrile; H₂O represents water; DMSO represents dimethyl sulfoxide; MeOH represents methanol; NH₄HCO₃ represents ammonium bicarbonate; LAH represents lithium aluminum hydride; BOC represents t-butoxycarbonyl, which is an amine-protective group; Ms represents methanesulfonyl, which is a protective group; TBS represents t-butyldimethylsilyl, which is a protective group; LDA represents lithium diisopropylamide; M represents mol/L; N/A represents undetectable; MgCl₂ represents magnesium chloride; EGTA represents ethylene glycol bis(2-aminoethyl)tetraacetic acid; Na₃VO₄ represents sodium vanadate.

Compounds are named according to conventional nomenclature rules in the art or using ChemDraw® software, and supplier's catalog names are given for commercially available compounds.

### BRIEF DESCRIPTION OF THE DRAWINGS

FIG. 1 illustrates the plasma TNF-alpha concentration in SD rats induced by lipopolysaccharide (LPS).

FIG. 2 illustrates the body weight change in different groups of human B-cell lymphoma OCI-LY10 cells subcutaneous xenograft tumor mouse models receiving the compound of the invention in an in vivo pharmacodynamic study.

FIG. 3 illustrates the relative weight change (%) of human B-cell lymphoma OCI-LY10 cells subcutaneous xenograft tumor mouse models receiving the compound of the invention in an in vivo pharmacodynamic study.

FIG. 4 illustrates tumor growth in human B-cell lymphoma OCI-LY10 cells subcutaneous xenograft tumor mouse models receiving the compound of the invention in an in vivo pharmacodynamic study.

FIG. 5 illustrates the body weight change in different groups of mice with collagen-induced arthritis in an in vivo pharmacodynamic study of the compound of the invention.

FIG. 6 illustrates the variation of clinical scores in different groups of mice with collagen-induced arthritis in an in vivo pharmacodynamic study of the compound of the invention.

FIG. 7 illustrates the area under the clinical score curve in different groups of mice with collagen-induced arthritis in an in vivo pharmacodynamic study of the compound of the invention.

### DETAILED DESCRIPTION

The present disclosure is described in detail below by way of examples. However, this is by no means disadvantageously limiting the scope of the invention. Although the invention has been described in detail herein and specific examples have also been disclosed, it will be apparent to those skilled in the art that various changes and modifications can be made to the specific examples without departing from the spirit and scope of the present disclosure.

### Intermediate A1

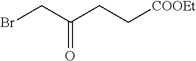 [SMILES: CCOC(=O)CCC(=O)CBr]

Synthetic Route:

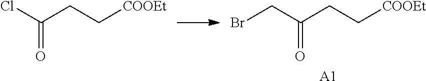

Step 1: Synthesis of Compound A1

Ethyl succinyl chloride (50 g) was added to acetonitrile (500 mL) and the mixture was stirred homogeneously. Trimethylsilyldiazomethane (2 M, 227.84 mL) was added dropwise to the mixture, and the mixture was stirred at 25° C. for 0.5 h. After the reaction system was cooled to 0° C., a solution of hydrobromic acid in acetic acid (93.10 g, 33%) was added dropwise to the reaction system. The reaction system was allowed to return to 25° C. and stirred for 0.5 h. The reaction was terminated, and acetonitrile was removed by concentration at reduced pressure. The remaining liquid was poured into 500 mL of ethyl acetate and washed with 100 mL of saturated sodium bicarbonate solution three times. The organic phase was separated and dried over an appropriate amount of anhydrous sodium sulfate.

The resulting reaction mixture was filtered to remove the desiccant, and the filtrate was concentrated at reduced pressure to give a crude product. The crude product was subjected to column purification (petroleum ether to petroleum ether:ethyl acetate =10:1) to give an intermediate A1.

Intermediates in Table 1 below are commercially available reagents.

TABLE 1

| No. | Structures | CAS |
|---|---|---|
| B1 | 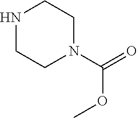 | 50606-31-0 |
| B2 | 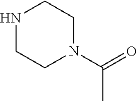 | 13889-98-0 |
| B3 | 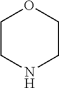 | 110-91-8 |
| B4 | 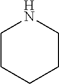 | 110-89-4 |
| B5 | 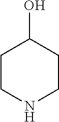 | 5382-16-1 |
| B6 | 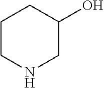 | 6859-99-0 |
| B7 | 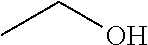 | 64-17-5 |
| B8 | 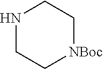 [SMILES: CC(C)(C)OC(=O)N1CCNCC1] | 57260-71-6 |
| B9 | 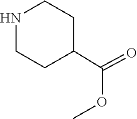 | 2971-79-1 |
| B10 | 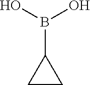 | 411235-57-9 |
| B11 | 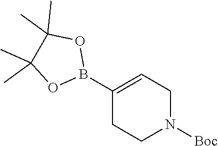 [SMILES: CC(C)(C)OC(=O)N1CC=C(B2OC(C)(C)C(C)(C)O2)CC1] | 286961-14-6 |
| B12 | 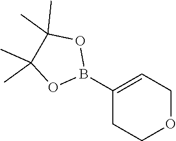 | 287944-16-5 |
| B13 | 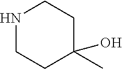 | 3970-68-1 |
| B14 | 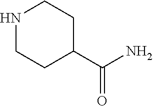 | 39546-32-2 |
| B15 | 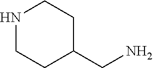 | 7144-05-0 |
| B16 | 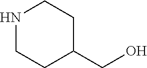 | 6457-49-4 |
| B17 | 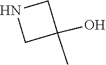 | 256931-54-1 |

### Example 1: Synthesis of Compound WX001

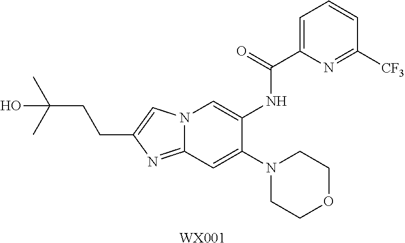

Synthetic Route:

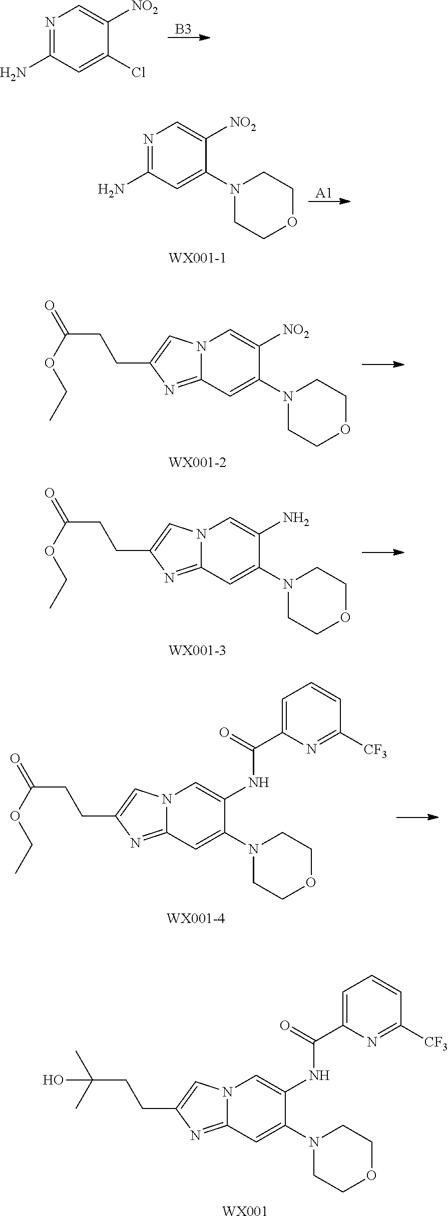

Step 1: Synthesis of Compound WX001-1

4-Chloro-5-nitro-pyridin-2-amine (0.2 g) was added to B3 (1.0 g), and the resulting mixture was stirred at 14° C. for 16 h. The reaction mixture was concentrated to dryness at reduced pressure, and 10 mL of ethyl acetate was added to the residue. The mixture was stirred for 10 min. Insoluble matters were removed by filtration, and the filtrate was concentrated at reduced pressure to give WX001-1. LCMS (ESI) m/z:=225.8 [M+H]⁺, ¹H NMR (400 MHz, CD₃OD) δ=8.55 (s, 1H), 5.97 (s, 1H), 3.85-3.80 (m, 4H), 3.12-3.06 (m, 4H).

Step 2: Synthesis of Compound WX001-2

Compound WX001-1 (0.1 g) was added to intermediate Al (129.33 mg), and the resulting mixture was stirred at 100° C. for 16 h. After the reaction mixture was cooled to room temperature, 10 mL of ethyl acetate and 5 mL of a saturated aqueous sodium bicarbonate were added to the reaction mixture. The mixture was stirred until the solid was completely dissolved. The organic phase was separated after standing and the aqueous phase was extracted with 10 mL of ethyl acetate twice. The organic phases were combined and dried over an appropriate amount of anhydrous sodium sulfate. The resulting reaction mixture was filtered to remove the desiccant, and the filtrate was concentrated at reduced pressure to give a crude product. The crude product was purified by column chromatography (eluent: methanol/ethyl acetate =0-10%) to give compound WX001-2. LCMS (ESI) m/z:=349.1 [M+H]⁺, ¹H NMR (400 MHz, MeOD-d4) δ=9.27 (s, 1H), 7.63 (s, 1H), 6.98 (s, 1H), 4.14 (q, J=6.8 Hz, 2H), 3.87-3.83 (m, 4H), 3.20-3.07 (m, 4H), 2.75-2.60 (m, 2H), 2.35-2.20 (m, 2H), 1.27-1.22 (m, 3H).

Step 3: Synthesis of Compound WX001-3

Compound WX001-2 (0.82 g) was dissolved in ethanol (10 mL) and Raney nickel (605.02 mg) was added in argon atmosphere. After being purged with argon three time and with hydrogen three times, the mixture was stirred at 50° C. for 16 h at 50 Psi in hydrogen atmosphere. After the reaction mixture was cooled to room temperature, the catalyst was removed by filtration through celite pad. The filtrate was concentrated under reduced pressure to obtain compound WX001-3. LCMS (ESI) m/z:=319.0 [M+H]⁺, ¹H NMR (400 MHz, DMSO-d6) δ=7.68 (s, 1H), 7.35 (s, 1H), 6.82 (s, 1H), 4.51 (s, 2H), 4.06 (q, J=8.0 Hz, 2H), 3.80-3.76 (m, 4H), 2.91-2.75 (m, 6H), 2.68-2.63 (m, 2H), 1.18 (t, J=7.0 Hz, 3H).

Step 4: synthesis of compound WX001-4

Compound WX001-3 (0.05 g) was dissolved in anhydrous dichloromethane (5 mL). 6-(Trifluoromethyl)pyridin-2-carboxylic acid (36.02 mg), O-(7-azabenzotriazol-1 -yl)-N,N,N′,N′-tetramethylurea hexafluorophosphate (89.57 mg), and N,N-diisopropylethylamine (40.59 mg) were added, and the resulting reaction solution was stirred at 10° C. for 3 h. The reaction solution was diluted with 10 mL of dichloromethane and then washed with water three times (10 mL each time). The organic phases were combined and washed with 10 mL of saturated brine. Then the organic phase was dried over an appropriate amount of anhydrous sodium sulfate, and filtered to remove the desiccant.

The filtrate was concentrated at reduced pressured to give compound WX001-4. LCMS (ESI) m/z:=492.1 [M+H]⁺.

Step 5: Synthesis of Compound WX001

Compound WX001-4 (48.42 mg) was dissolved in anhydrous tetrahydrofuran (5 mL), before methylmagnesium bromide in ethyl ether (3M, 164.22 μL) was added at 10° C. The mixture was stirred at 10° C. for 10 min. 2 mL of saturated aqueous ammonium chloride and 5 mL of water were added to the reaction mixture to quench the reaction. The tetrahydrofuran layer was separated and the aqueous phase was extracted with ethyl acetate three times (10 mL each time). The organic phases were combined and dried over an appropriate amount of anhydrous sodium sulfate. The resulting reaction mixture was filtered to remove the desiccant, and the filtrate was concentrated at reduced pressure to give a crude product. The crude product was subjected to separation by high pressure liquid chromatography HPLC (column: Boston Green ODS 150×30, 5 μm; mobile phase: A: 0.1% trifluoroacetic acid in water, B: acetonitrile; gradient: B%: 25%-55%, 8 min) and supercritical fluid chromatography SFC (column: DAICEL CHIRALPAK IC (250 mm×30 mm, 10 μm); mobile phase: A: 0.1% ammonia in ethanol, B: liquid carbon dioxide; gradient: B%: 50%-50%) to give compound WX001. LCMS (ESI) m/z=478.1[M+H]⁺, ¹H NMR (400 MHz, DMSO-d₆) δ=10.81 (brs, 1H), 9.71 (s, 1H), 8.62-8.52 (m, 2H), 8.45 (d, J=6.8 Hz, 1H), 7.95 (s, 1H), 7.52 (s, 1H), 4.62(s, 1H), 4.13-3.94 (m, 4H), 3.20-3.02 (m, 4H), 2.90-2.98 (m, 2H), 2.01-1.90 (m, 2H), 1.34 (s, 6H).

### Example 2: Synthesis of Compound WX002

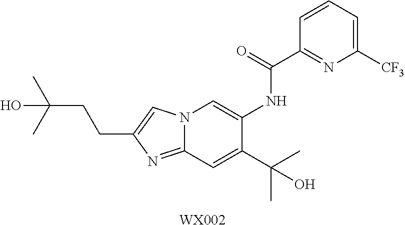

Synthetic Route:

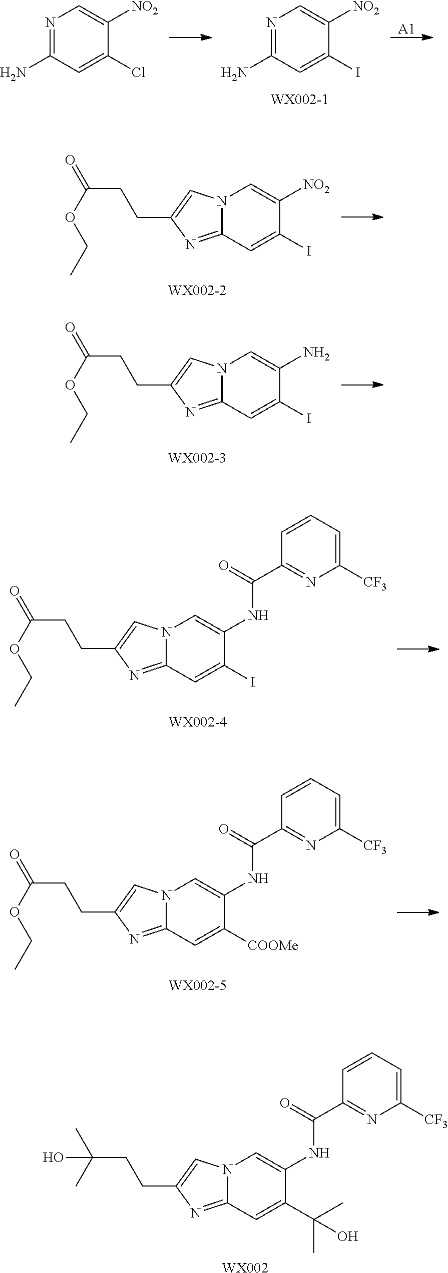

Step 1: Synthesis of Compound WX002-1

2-Butanone (510 mL) and 2-amino-4-chloro-5-nitropyridine (30 g) were stirred homogeneously. Sodium iodide (77.73 g) and hydroiodic acid (29.14 g) were added to the reaction system, which was then warmed to 84° C. for reacting 24 h. The reaction solution was cooled to room temperature, and concentrated at reduced pressure to about 250 mL, to which was added with 500 mL of water, and the mixture was stirred for 15 min. The reaction solution was filtered to give a crude product. 6 g of sodium thiosulfate was dissolved in 120 mL of water before the above crude product was added. The mixture was stirred for 30 min and filtered. The filter cake was rinsed with water 3 times (60 mL each time) and dried to give compound WX002-1. LCMS (ESI) m/z=265.9 [M+H]⁺.

Step 2: Synthesis of Compound WX002-2

Compound WX002-1 (25 g) was added to intermediate A1 (29.46 g), and the resulting mixture was stirred at 100° C. for 12 h. After the reaction mixture was cooled to room temperature, an appropriate amount of methanol was added to the reaction mixture. The mixture was stirred until the solid was completely dissolved. The methanol solution was concentrated to dryness at reduced pressure to give a brown viscous solid. The brown viscous solid was mixed with 50 mL of ethyl acetate. The mixture was stirred for 30 min and filtered. The filter cake was mixed with 50 mL of ethyl acetate before the mixture was stirred for 30 min and filtered again. The filter cake was dried to give compound WX002-2. LCMS (ESI) m/z=390.0 [M+H]⁺.

Step 3: Synthesis of Compound WX002-3

Compound WX002-2 (6 g) was added to ethanol (100 mL) and stirred homogeneously. Aqueous ammonium chloride solution (4 M, 30.00 mL) and iron powder (2.15 g) were added to the reaction system. The system was warmed to 90° C. and stirred for 1 h. The mixture was hot filtered, and the filter cake was washed thoroughly with methanol 3 times (50 mL each). The filtrates were combined and concentrated at reduced pressure to give a crude product. The crude product was purified by column chromatography (dichloromethane:methanol=100:0-70:30) to give compound WX002-3. LCMS (ESI) m/z=360.0 [M+H]⁺.

Step 4: Synthesis of Compound WX002-4

Compound WX002-3 (1.4 g) was added to N,N-dimethylformamide (14 mL) and stirred homogeneously. O-(7-azabenzotriazol-1-yl)-N,N,N′,N′-tetramethylurea hexafluorophosphate (2.22 g), 6-trifluoromethylpyridine-2-carboxylic acid (819.42 mg) and N,N-diisopropylethylamine (1.51 g) were added, and the resulting reaction solution was reacted at room temperature and 15° C. for 2 h. The reaction solution was filtered, and the filter cake was washed thoroughly with N,N-dimethylformamide (2 mL) and dried to give compound WX002-4. LCMS (ESI) m/z=533.1 [M+H]⁺.

Step 5: Synthesis of Compound WX002-5

Compound WX002-4 (500 mg) was added to methanol (20 mL), before 2-dicyclohexylphosphino-2,4,6-triisopropylbiphenyl (463.80 mg), palladium acetate (42.18 mg) and triethylamine (285.17 mg) were added successively. The resulting reaction mixture was reacted at 50 Psi in carbon monoxide at 80° C. for 13 h. The reaction liquid was cooled to room temperature and filtered through celite pad. The filter cake was washed with methanol twice (10 mL each). The filtrates were combined and concentrated at reduced pressure to dryness to give a crude product of WX002-5 without further purification.

Step 6: Synthesis of Compound WX002

The crude product WX002-5 (250 mg) was dissolved in anhydrous tetrahydrofuran (2.5 mL), and the mixture was cooled to 0° C. Methylmagnesium bromide in ethyl ether (3 M, 1.44 mL) was slowly added dropwise and the mixture was stirred for 2 h. The reaction was quenched by adding 2 mL of 1 M diluted hydrochloric acid to the reaction mixture. The organic phase was separated and the aqueous phase was extracted with ethyl acetate three times (2 mL each). The organic phases were combined, washed with 3 mL of saturated brine, and dried over anhydrous sodium sulfate. The resulting reaction mixture was filtered to remove the desiccant, and the filtrate was concentrated at reduced pressure to give a crude product. The crude product was separated by high pressure liquid chromatography HPLC (column: Welch Xtimate C18 150×25 mm×5 μm; mobile phase: A: 10 mM NH₄HCO₃ in water, B: methanol; gradient: B%: 52%-72%, 10.5 min) to give compound WX002. LCMS (ESI) m/z=451.3 [M+H]⁺, 1H NMR (400 MHz, CDC13) δ=12.18 (s, 1H), 9.56 (s, 1H), 8.46-8.51 (m, 1H), 8.13 (t, J=7.84, 1H), 7.87 (d, J=8.0, 1H), 7.45 (s, 1H), 7.35 (s, 1H), 3.92 (s, 1H), 2.87-2.98 (m, 2H), 2.64 (s, 1H), 1.96 (t, J=7.60, 2H), 1.75 (s, 6H), 1.33 (s, 6H).

Reference to Example 1 was made for the synthesis procedures except that B3 (morpholine) in step 1 in Example 1 was replaced by a corresponding B fragment in the corresponding fragment 1. The synthesis procedures might comprise cleaving Boc, hydrolysis or hydrogenation operations, etc. The final synthesis of the examples are shown in Table 2 below.

TABLE 2

| Example | Fragment 1 | Compound | Product structure | NMR & LCMS |
|---|---|---|---|---|
| 3 | B4 | WX003 | 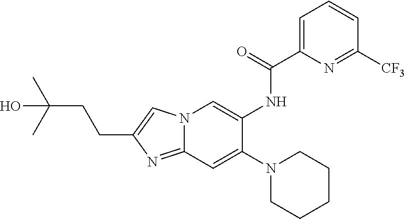 [SMILES: CC(C)(O)CCc1cn2cc(NC(=O)c3cccc(C(F)(F)F)n3)c(N3CCCCC3)cc2n1] | ¹H NMR (400 MHz, CD₃OD) δ = 9.42 (s, 1H), 8.40 (d, J = 7.6 Hz, 1H), 8.23 (t, J = 8.0 Hz, 1H), 7.99 (d, J = 7.6 Hz, 1H), 7.45 (s, 1H), 7.07 (s, 1H), 2.86-2.85 (m, 4H), 2.73-2.69 (m, 2H), 1.82-1.78 (m, 6H), 1.59 (br s, 2H), 1.18 (s, 6H). LCMS (ESI) m/z = 476.1 [M + H]⁺ |
| 4 | B2 | WX004 | 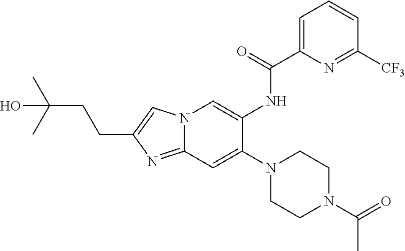 [SMILES: CC(=O)N1CCN(c2cc3nc(CCC(C)(C)O)cn3cc2NC(=O)c2cccc(C(F)(F)F)n2)CC1] | ¹H NMR (400 MHz, CD₃OD) δ = 9.59 (s, 1H), 8.55 (d, J = 8.0 Hz, 1H), 8.37 (t, J = 8.4 Hz, 1H), 8.13 (d, J = 7.6 Hz, 1H), 7.64 (s, 1H), 7.29 (s, 1H), 3.91-3.88 (m, 4H), 3.11-3.04 (m, 4H), 2.87-2.83 (m, 2H), 2.21 (s, 3H), 1.94-1.90 (m, 2H), 1.30 (s, 6H). LCMS (ESI) m/z = 519.3 [M + H]⁺ |
| 5 | B5 | WX005 | 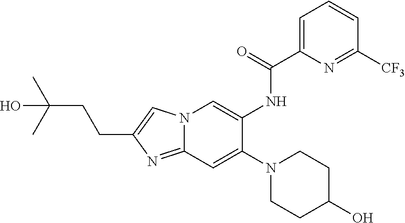 [SMILES: CC(C)(O)CCc1cn2cc(NC(=O)c3cccc(C(F)(F)F)n3)c(N3CCC(O)CC3)cc2n1] | ¹H NMR (CDCl₃) δ = 10.52 (1H, s), 9.65 (1H, s), 8.52 (d, J = 4.0 Hz, 1H), 8.22 (t, J = 8.0 Hz, 1H), 7.97 (d, J = 7.6 Hz, 1H), 7.75- 7.68 (m, 1H), 7.37 (s, 1H), 4.14- 4.12 (m, 1H), 3.25-3.15 (m, 1H), 3.07-2.80 (m,6H), 1.78-2.09 (m, 6H), 1.60-1.48 (m, 1H), 1.28 (s, 6H). LCMS (ESI) m/z = 492.1 [M + H]⁺ |
| 6 | B6 | WX006 | 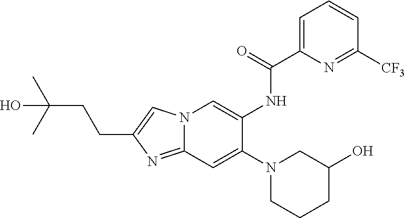 [SMILES: CC(C)(O)CCc1cn2cc(NC(=O)c3cccc(C(F)(F)F)n3)c(N3CCCC(O)C3)cc2n1] | ¹H NMR (CDCl₃) δ = 10.52 (1H, s), 9.65 (1H, s), 8.51 (d, J = 4.0 Hz, 1H), 8.22 (t, J = 8.0 Hz, 1H), 7.97 (d, J = 7.6 Hz, 1H), 7.82- 7.79 (m, 1H), 7.35 (s, 1H), 4.14- 4.12 (m, 1H), 3.38-3.10 (m, 1H), 3.06-2.87 (m, 6H), 2.05-1.95 (m, 6H), 1.60-1.48 (m, 1H), 1.29 (s, 6H). LCMS (ESI) m/z = 492.1 [M + H]⁺ |
| 7 | B16 | WX007 | 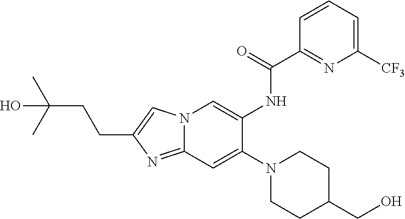 [SMILES: CC(C)(O)CCc1cn2cc(NC(=O)c3cccc(C(F)(F)F)n3)c(N3CCC(CO)CC3)cc2n1] | ¹H NMR (400 MHz, CD₃OD) δ = 9.64 (s, 1H), 8.48 (br d, J = 7.8 Hz, 1H), 8.32 (br t, J = 7.6 Hz, 2H), 8.08 (d, J = 7.6 Hz, 1H), 7.74 (s, 1H), 7.36 (s, 1H), 3.50 (d, J = 6.4 Hz, 2H), 2.92-2.73 (m, 5H), 2.02-1.84 (m, 4H), 1.70 (br d, J = 7.6 Hz, 1H), 1.64- 1.48 (m, 2H), 1.26 (s, 6H). LCMS (ESI) m/z = 506.1 [M + H]⁺ |
| 8 | B8 | WX008 | 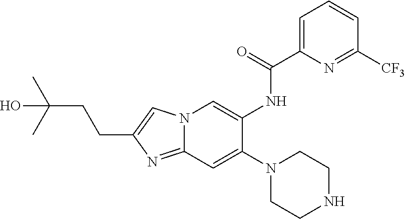 [SMILES: CC(C)(O)CCc1cn2cc(NC(=O)c3cccc(C(F)(F)F)n3)c(N3CCNCC3)cc2n1] | LCMS (ESI) m/z = 477.0 [M + H]⁺ |
| 9 | B9 | WX009 | 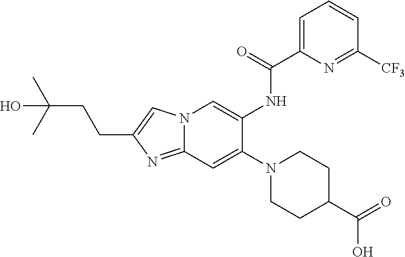 [SMILES: CC(C)(O)CCc1cn2cc(NC(=O)c3cccc(C(F)(F)F)n3)c(N3CCC(C(=O)O)CC3)cc2n1] | LCMS (ESI) m/z = 520.1 [M + H]⁺ |
| 10 | B1 | WX010 | 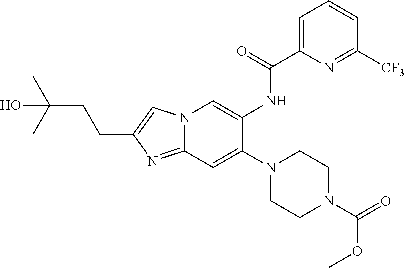 [SMILES: COC(=O)N1CCN(c2cc3nc(CCC(C)(C)O)cn3cc2NC(=O)c2cccc(C(F)(F)F)n2)CC1] | LCMS (ESI) m/z = 535.1 [M + H]⁺ |
| 11 | B12 | WX011 | 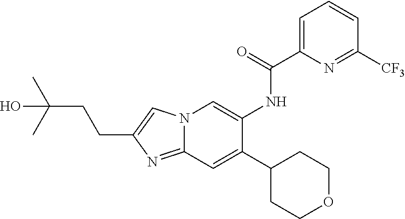 [SMILES: CC(C)(O)CCc1cn2cc(NC(=O)c3cccc(C(F)(F)F)n3)c(C3CCOCC3)cc2n1] | ¹H NMR (400 MHz, CDCl₃) δ = 10.03 (s, 1H), 9.27 (s, 1H), 8.44 (d, J = 8.0 Hz, 1H), 8.14- 8.11 (m, 1H), 7.87 (d, J = 7.2 Hz, 1H), 7.37 (s, 1H), 7.29 (s, 1H), 4.09 (d, J = 10.8 Hz, 2H), 3.58 (brs, 2H), 3.01-2.84 (m, 3H), 2.11-1.90 (m, 7H), 1.26 (s, 6H). LCMS (ESI) m/z = 477.1 [M + H]⁺ |
| 12 | B11 | WX012 | 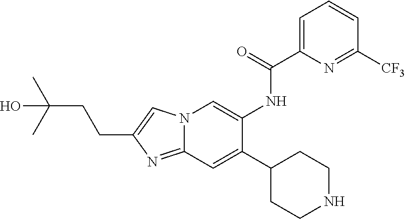 [SMILES: CC(C)(O)CCc1cn2cc(NC(=O)c3cccc(C(F)(F)F)n3)c(C3CCNCC3)cc2n1] | ¹H NMR (400 MHz, CD₃OD) δ = 9.19 (s, 1H), 8.50 (d, J = 8.0 Hz, 1H), 8.38 (t, J = 8.0 Hz, 1H), 8.15 (d, J = 8.0 Hz, 1H), 8.02 (s, 1H), 7.88 (s, 1H) 3.59 (d, J = 12.0 Hz, 2H), 3.38-3.51 (m, 1H), 3.14 (t, J = 13.2 Hz, 2H), 3.04-2.99 (m, 2H), 2.68-2.34 (m, 2H), 2.16- 2.10 (m, 2H), 1.98-1.94 (m, 2H), 1.31(s, 6H). LCMS (ESI) m/z = 476.2 [M + H]⁺ |
| 13 | B10 | WX013 | 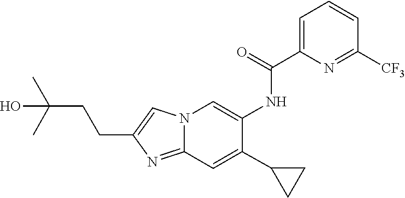 [SMILES: CC(C)(O)CCc1cn2cc(NC(=O)c3cccc(C(F)(F)F)n3)c(C3CC3)cc2n1] | ¹H NMR (400 MHz, CD₃OD) δ = 9.73 (br s, 1H), 8.54 (d, J = 7.2 Hz, 1H), 8.38 (br s, 1H), 8.14 (d, J = 6.8 Hz, 1H), 8.01 (br s, 1H), 7.64 (brs, 1H), 3.08-2.98 (m, 1H), 2.21 (br s, 2H), 1.94 (br s, 2H), 1.34 (br s, 2H), 1.30 (s, 6H), 0.99 (br s, 2H). LCMS (ESI) m/z = 433.1 [M + H]⁺ |
| 14 | B7 | WX014 | 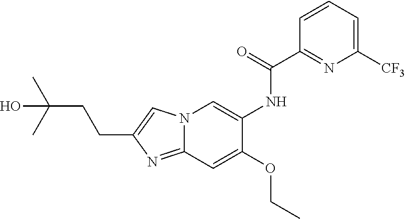 [SMILES: CCOc1cc2nc(CCC(C)(C)O)cn2cc1NC(=O)c1cccc(C(F)(F)F)n1] | ¹H NMR (400 MHz, CD₃OD) δ = 9.70 (s, 1H), 8.51 (d, J = 8.0 Hz, 1H), 8.36 (t, J = 8.0 Hz, 1H), 8.12 (d, J = 7.2, 1H), 7.78 (s, 1H), 7.20 (s, 1H), 4.44-4.39 (m, 2H), 2.93- 2.87 (m, 2H), 1.94-1.90 (m, 2H), 1.65 (t, J = 6.8 Hz, 3H), 1.30 (s, 6H). LCMS (ESI) m/z = 437.0 [M + H] |
| 15 | B13 | WX015 | 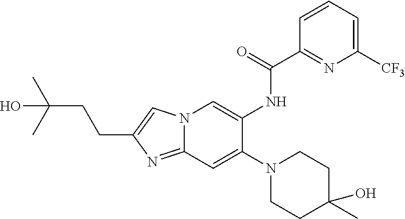 [SMILES: CC(C)(O)CCc1cn2cc(NC(=O)c3cccc(C(F)(F)F)n3)c(N3CCC(C)(O)CC3)cc2n1] | ¹H NMR(400 MHz, CD₃OD) δ = 9.46 (s, 1H), 8.45 (br d, J = 7.8 Hz, 1H), 8.30 (br t, J = 7.8 Hz, 1H), 8.06 (br d, 7 = 7.8 Hz, 1H), 7.49 (s, 1H), 7.18 (s, 1H), 3.10 (br t, J = 10.8 Hz, 2H),2.91 (brd, 7 = 11.2 Hz, 2H), 2.83-2.73 (m, 2H), 2.09-1.84 (m, 4H), 1.75 (br d, J = 13.2 Hz, 2H), 1.35- 1.20 (m, 9H). LCMS (ESI) m/z = 506.1 [M + H]⁺ |
| 16 | B14 | WX016 | 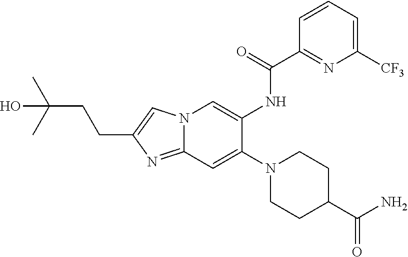 [SMILES: CC(C)(O)CCc1cn2cc(NC(=O)c3cccc(C(F)(F)F)n3)c(N3CCC(C(N)=O)CC3)cc2n1] | ¹H NMR (400 MHz, CD₃OD) δ = 10.51 (s, 1H), 9.49 (s, 1H), 8.52-8.36 (m, 2H), 8.26-8.17 (m, 2H), 7.71 (s, 1H), 7.23 (br s, 2H), 6.79 (br s, 1H), 3.12 (br d, J = 11.6 Hz, 1H), 3.17-3.06 (m, 1H), 2.79-2.65 (m, 4H), 2.36- 2.21 (m, 1H), 1.98-1.83 (m, 4H), 1.79-1.68 (m, 2H), 1.14 (s, 6H). LCMS (ESI) m/z = 519.1 [M + H]⁺ |
| 17 | B15 | WX017 | 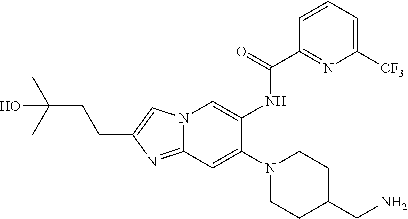 [SMILES: CC(C)(O)CCc1cn2cc(NC(=O)c3cccc(C(F)(F)F)n3)c(N3CCC(CN)CC3)cc2n1] | ¹H NMR (400 MHz, DMSO-d₆) δ = 10.62 (br s, 1H), 9.50 (s, 1H), 8.54-8.39 (m, 2H), 8.24 (br d, J = 7.2 Hz, 1H), 7.72 (s, 1H), 7.25 (s, 1H), 3.10 (br d, J = 10.0 Hz, 1H), 2.75-2.65 (m, 4H), 2.33 (br s, 2H), 2.05-1.87 (m, 5H), 1.79-1.72 (m, 2H), 1.41 (br s, 2H), 1.15 (s, 6H), 0.86 (br s, 2H). LCMS (ESI) m/z = 505.1 [M + H]⁺ |

### Example 18: Synthesis of Compound WX018

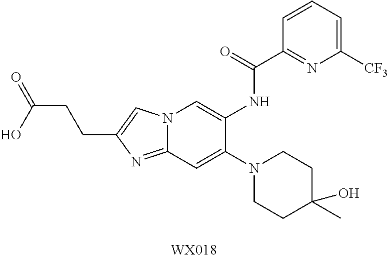

Synthetic Route:

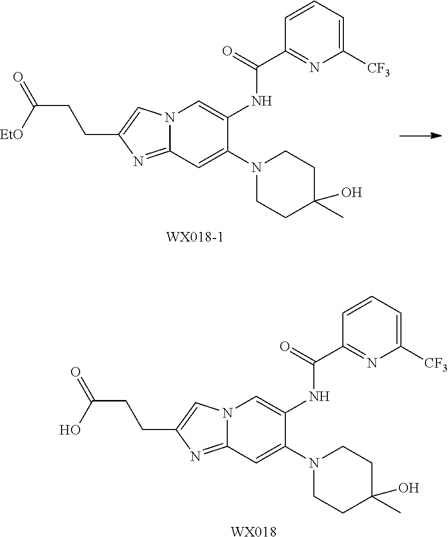

Step 1: Synthesizing WX018-1 with Reference to the Synthesis of Compound WX001

Step 2: Synthesis of Compound WX018

Compound WX018-1 (550 mg) was added to a mixed solution of tetrahydrofuran (10.0 mL) and water (10.0 mL) before sodium hydroxide (254.06 mg) was added. The resulting mixture was stirred at 30° C. for 16 h. Tetrahydrofuran was removed by concentration at reduced pressure, and 1 M diluted hydrochloric acid was added dropwise with stirring until the solution reach about pH 3. A solid was precipitated. The mixture was filtered and the filter cake was collected. The filter cake was purified by high performance liquid chromatography HPLC [column: YMC Triart C18 150×25 mm×5 μm; mobile phase: [H₂O (10 mM NH₄HCO₃)—ACN]; B% gradient: 21%-51%, 9.5 min], and lyophilized to give compound WX018.

¹H NMR (400 MHz, DMSO-d₆) δ=10.59 (s, 1H), 9.49 (s, 1H), 8.54-8.34 (m, 2H), 8.23 (d, J=7.8 Hz, 1H), 7.73 (s, 1H), 7.23 (s, 1H), 4.40 (br s, 1H), 3.03 (br t, J=10.8 Hz, 2H), 2.92-2.76 (m, 5H), 2.63 (brt, J=7.6 Hz, 2H), 1.90-1.75 (m, 2H), 1.65 (brd, J=12.4 Hz, 2H), 1.23 (s, 3H). LCMS (ESI) m/z=492.1[M+H]⁺

Referring to the synthesis procedures of Example 1 and Example 18, examples in Table 3 below were synthesized starting from the corresponding B fragment in Fragment 1 in the following table.

TABLE 3

| Example | Fragment 1 | Compound | Product structure | NMR |
|---|---|---|---|---|
| 19 | B14 | WX019 | 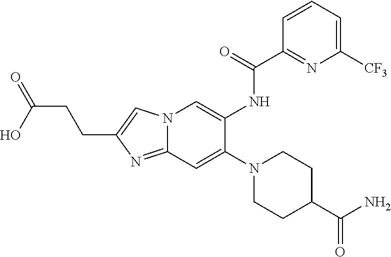 [SMILES: NC(=O)C1CCN(c2cc3nc(CCC(=O)O)cn3cc2NC(=O)c2cccc(C(F)(F)F)n2)CC1] | ¹H NMR (400 MHz, DMSO-d₆) δ = 10.51 (s, 1H), 9.50 (s, 1H), 8.62-8.35 (m, 2H), 8.22 (br d, J = 7.6 Hz, 1H), 7.74 (s, 1H), 7.36- 7.12 (m, 2H), 6.78 (br s, 1H), 3.12 (br d, J = 10.8 Hz, 1H), 2.87 (br t, J = 6.8 Hz, 2H), 2.77-2.65 (m, 3H), 2.37-2.20 (m, 2H), 1.97- 1.81 (m,4H), 1.23 (br s, 2H). LCMS (ESI) m/z = 505.4 [M + H]⁺ |
| 20 | B15 | WX020 | 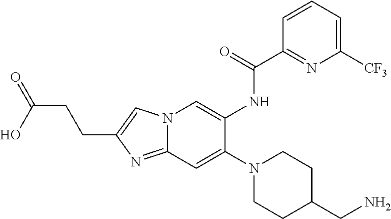 [SMILES: NCC1CCN(c2cc3nc(CCC(=O)O)cn3cc2NC(=O)c2cccc(C(F)(F)F)n2)CC1] | ¹H NMR; (400 MHz, DMSO-d₆) δ = 10.73-10.39 (m, 1H), 9.51- 9.33 (m, 1H), 8.54-8.12 (m, 3H), 7.62 (br d, J = 15.2 Hz, 1H), 7.32- 7.04 (m, 1H), 3.05 (br d, J = 10.8 Hz, 1H), 2.91-2.75 (m, 4H), 2.67 (br s, 1H), 2.35-2.22 (m, 2H), 2.07-1.94 (m, 1H), 1.86 (br s, 1H), 1.69 (br d, J = 12.4 Hz, 1H), 1.39 (br s, 2H), 1.23 (brs, 5H). LCMS (ESI) m/z = 491.1 [M + H]⁺ |
| 21 | B16 | WX021 | 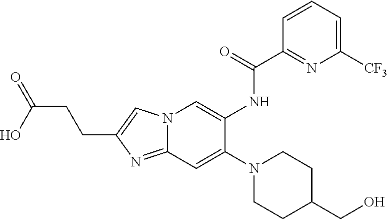 [SMILES: O=C(O)CCc1cn2cc(NC(=O)c3cccc(C(F)(F)F)n3)c(N3CCC(CO)CC3)cc2n1] | ¹H NMR (400 MHz, DMSO-d₆) δ = 10.41 (s, 1H), 9.75 (s, 1H), 8.60-8.38 (m, 1H), 8.60-8.38 (m, 1H), 8.27 (d, J = 7.6 Hz, 1H), 8.13 (s, 1H), 7.46 (s, 1H), 4.63 (br s, 1H), 3.34 (br d, J = 6.6 Hz, 2H), 3.25 (br d, J = 11.6 Hz, 2H), 3.01 (br t, J = 7.3 Hz, 2H), 2.86-2.68 (m, 4H), 1.89 (br d, J = 11.4 Hz, 2H), 1.62 (br s, 1H), 1.51-1.33 (m, 3H). LCMS (ESI) m/z = 492.0 [M + H]⁺ |
| 22 | B17 | WX022 | 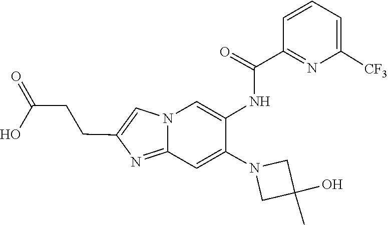 [SMILES: CC1(O)CN(c2cc3nc(CCC(=O)O)cn3cc2NC(=O)c2cccc(C(F)(F)F)n2)C1] | ¹H NMR (400 MHz, DMSO-d₆) δ = 9.97 (s, 1H), 8.86 (s, 1H), 8.45- 8.40 (m, 2H), 8.24 (br d, J = 7.2 Hz, 1H), 8.18 (s, 1H), 7.53 (s, 1H), 6.52 (s, 1H), 3.90 (br d, J = 7.4 Hz, 2H), 3.73 (br d, J = 7.4 Hz, 3H), 2.88-2.83 (m, 2H), 2.62 (br t, J = 7.3 Hz, 2H), 1.49 (s, 3H). LCMS (ESI) m/z = 464.1 [M + H]⁺ |
| 24 | B5 | WX024 | 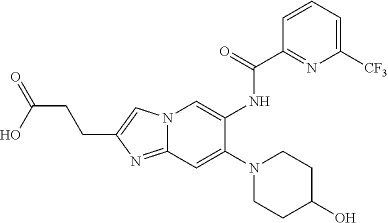 [SMILES: O=C(O)CCc1cn2cc(NC(=O)c3cccc(C(F)(F)F)n3)c(N3CCC(O)CC3)cc2n1] | ¹H NMR (400 MHz, DMSO-d₆) δ = 10.59 (s, 1H), 9.50 (s, 1H), 8.54- 8.37 (m, 2H), 8.24 (d, J = 7.8 Hz, 1H), 7.74 (s, 1H), 7.26 (s, 1H), 4.76 (br s, 1H), 3.75 (br s, 1H), 3.05 (br d, J = 5.2 Hz, 2H), 2.87 (br t, J = 7.4 Hz, 2H), 2.77 (br t, J = 8.9 Hz, 2H), 2.70-2.55 (m, 3H), 1.91 (br s, 2H), 1.71 (br d, J = 8.6 Hz, 2H). LCMS (ESI) m/z = 478.1 [M + H]⁺ |

### Example 23: Synthesis of Compound WX023

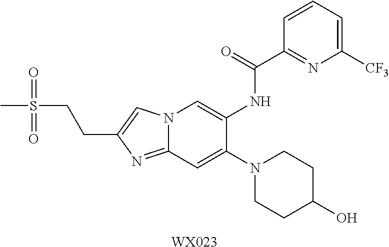

Synthetic Route:

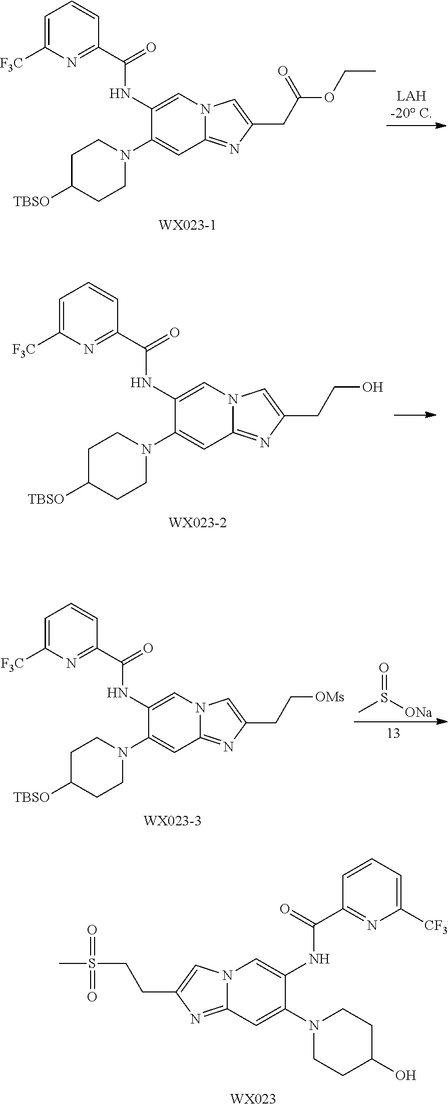

Step 1: Reference was made to the synthesis of compound WX001 using fragment B5 as the starting material, and after the synthetic procedures using TB SC1 to protect hydroxyl, intermediate WX023-1 was obtained.

Step 2: Synthesis of Compound WX023-2

Tetrahydrofuran (30.0 mL) was added to lithium aluminum hydride (106.5 mg), and the mixture was cooled to 0° C. in nitrogen atmosphere. A solution of compound WX023-1 (1.7 g) in tetrahydrofuran (30.0 mL) was slowly added dropwise. The obtained mixed solution was stirred for 1 h at a temperature of −20° C. to 0° C. The reaction mixture was quenched by slowly pouring the mixture into 50.0 mL of saturated aqueous ammonium chloride with stirring at 0° C. The phases were separated and the aqueous phase was extracted with dichloromethane (100 mL×2). The organic phases were combined, dried, filtered and concentrated at reduced pressure to give compound WX023-2.

Step 3: synthesis of compound WX023-3

Trichloromethane (15.0 mL) was added to compound WX023-2 (1.2 g) before triethylamine (646.2 mg) was added. The resulting mixture was stirred in nitrogen atmosphere at 0° C. for 10 min. A solution of methanesulfonyl chloride (1.2 g) in trichloromethane (15.0 mL) was added dropwise. The mixture was allowed to naturally warm to 25° C., and stirred for 20 min. The tail gas was absorbed by aqueous saturated sodium bicarbonate solution. After the starting materials were completely reacted, the mixture was concentrated at a low temperature to give compound WX023-3.

Step 4: synthesis of compound WX023

N,N-dimethylformamide (5.0 mL) was added to compound WX023-3 (1.0 g) before sodium methanesulfinate (286.3 mg) and potassium iodide (776.0 mg) were added. The resulting mixture was reacted in microwave at 80° C. for 1 h. Four batches of rection mixture with same specification were combined. 20 mL of acetonitrile was added, and the mixture was suctioned under reduced pressure. The filtrate was concentrated at reduced pressure. The crude product was separated and purified by column chromatography (methanol=0-40%, dichloromethane:methanol) to give compound WX023. ¹H NMR (400 MHz, DMSO-d6) δ=10.55 (s, 1H), 9.59 (s, 1H), 8.55-8.38 (m, 2H), 8.25 (d, J=7.6 Hz, 1H), 7.94 (s, 1H), 7.33 (s, 1H), 4.79 (br s, 1H), 4.85-4.67 (m, 1H), 3.76 (br s, 1H), 3.57-3.46 (m, 2H), 3.21-3.07 (m, 5H), 3.03 (s, 3H), 1.98-1.89 (m, 2H), 1.80-1.64 (m, 2H). LCMS (ESI) m/z=512.1 [M+H]⁺

Referring to the synthesis procedures of Example 1 and Example 23, examples in Table 4 below was synthesized starting from Fragment 1 in the following table.

TABLE 4

| Example | Fragment 1 | Compound | Product structure | NMR |
|---|---|---|---|---|
| 25 | B13 | WX025 | 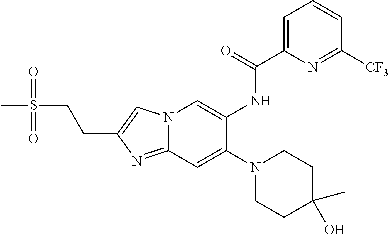 [SMILES: CC1(O)CCN(c2cc3nc(CCS(C)(=O)=O)cn3cc2NC(=O)c2cccc(C(F)(F)F)n2)CC1] | ¹H NMR (400 MHz, CDCl₃) δ = 10.50 (s, 1H), 9.61 (s, 1H), 8.47 (d, J = 7.8 Hz, 1H), 8.15 (t, .J = 7.8 Hz, 1H), 7.91 (d, J = 8.0 Hz, 1H), 7.67 (s, 1H), 7.41 (s, 1H), 5.28 (br s, 1H), 3.61-3.55 (m, 2H), 3.44-3.38 (m, 2H), 3.22- 3.12 (m, 2H), 2.95 (s, 3H), 2.19- 2.13 (m, 1H), 1.91-1.91 (m, 1H), 1.80-1.74 (m, 2H), 1.74- 1.67 (m, 2H), 1.26 (s, 3H). LCMS (ESI) m/z = 526.1 [M + H]⁺ |

Experimental Example 1: In Vitro Enzymatic Activity Evaluation

The inhibitory activity of the test compounds against human IRAK4 was evaluated by determining IC₅₀ values in a ³³P-labeled kinase activity assay (Reaction Biology Corp).

Buffer conditions: 20 mM Hepes (pH 7.5), 10 mM MgCl₂, 1 mM EGTA, 0.02% Brij35, 0.02 mg/mL BSA, 0.1 mM Na₃VO₄, 2 mM DTT, 1% DMSO.

Procedures: The test compound was dissolved in DMSO at room temperature to prepare a 10 mM solution for later use. The substrates were dissolved in freshly formulated buffers. The kinase was added, and mixture was stirred homogeneously. The DMSO solution containing the test compound was added to the above mixed reaction system by an acoustic technique (Echo 550). After 15 minutes of incubation, the reaction was started by adding ³³P-ATP. After 120 minutes of reaction at room temperature, the resulting solution was loaded on P81 ion exchange chromatography paper sheet (Whatman #3698-915). After repeated washings with 0.75% phosphoric acid solution, the radioactivity of the phosphorylated substrate residue on the paper sheet was measured. The kinase activity data are shown as a comparison of the kinase activity of the test compound and the kinase activity of the blank (DMSO only) and a curve was fitted using Prism4 software (GraphPad) to give IC₅₀ values, with the experimental results shown in Table 5.

TABLE 5
Results of in vitro kinase activity screening for
compounds disclosed herein

| | IRAK4/IC₅₀ |
|---|---|
| Compound | (nM) |
| WX001 | 2.2 |
| WX002 | 0.4 |
| WX003 | 1.2 |
| WX004 | 4.6 |
| WX005 | 1.3 |
| WX006 | 3.8 |
| WX007 | 0.9 |
| WX008 | 1.5 |
| WX010 | 4.6 |
| WX013 | 3.6 |
| WX014 | 1.7 |
| WX015 | 0.7 |
| WX016 | 1.2 |
| WX017 | 1.3 |
| WX018 | 9.8 |
| WX019 | 19.4 |
| WX020 | 20.5 |
| WX021 | 7.1 |
| WX023 | 1.3 |
| WX025 | 0.5 |

Conclusion: The compounds of the invention generally exhibit relatively good inhibitory activity against IRAK4.

### Experimental Example 2: In Vitro Activity Assay in Cells

TNF-α ELISA in THP-1 cells

1. Materials:

THP-1 human acute unicellular leukemia cells were purchased from ATCC (Cat # TIB-202) and incubated in a5% CO₂ incubator at 37° C. The medium was RPMI1640 (Gibco, Cat # 22400-105), the supplementary was 10% FBS (Gibco, Cat # 10091148); 1% PenStrep (Gibco, Cat # 15140); 0.05 mM 2-mercaptoethanol (Sigma, Cat # M6250).

2. Procedures:

TNF-α content in the cell culture supernatant was measured by a TNF-α Elisa kit. TNF-α was produced by stimulation of THP-1 cells with 150 ng/mL LPS (Sigma, Cat # L6529). Normal THP-1 cells in logarithmic phase were seeded in 96-well plates (Corning #3599) at a certain concentration (1×10⁵/100 μL) and then incubated in an incubator. After two hours, 16.7 μL of test compound of different concentrations (8×final concentration) was added and the mixture was incubated in the incubator. After one hour, 16.7 μL of 1200 ng/mL LPS was added and the mixture was incubated in the incubator. After 18 h, the culture was centrifuged and the probe of the supernatant was collected. The TNF-α content was measured by a TNF-α Elisa kit. Finally, the OD signals (OD450-OD570) were read on an envision plate reader.

3. Data analysis:

The OD450-OD570 signals were converted to percent inhibition.

Inhibition%=(ZPE−sample)/(ZPE−HPE)×100.

“HPE” represents the OD450-OD570 signal value of the control well without LPS-stimulated cells, and “ZPE” represents the OD450-OD570 signal value of the control well with LPS-stimulated cells.

IC₅₀ values for compounds were calculated by XLFit in the Excel.

Equation:y=Bottom+(Top-Bottom)/(1+(IC₅₀/X)^ HillSlope).

The test results are summarized in Table 6.

TABLE 6
Results of in vitro screening for compounds of the invention

| | THP-1/IC₅₀ |
|---|---|
| Compound | (nM) |
| WX002 | 54 |
| WX005 | 114 |
| WX015 | 201 |
| WX016 | 181 |

Conclusion: The compounds of the invention generally exhibit superior activity for inhibiting TNF-alpha generation in cells in a THP-1 cell activity assay.

### Experimental Example 3: Pharmacodynamic Study Evaluating Lipopolysaccharide (LPS)-Induced TNF-α Secretion in SD Rats 1. Modeling and Administration

SD rats were orally administered with a solvent, dexamethasone (DEX, 0.5 mg/kg) as positive control, and the test compound, and were intraperitoneally injected with LPS (1 mg/kg) 0.5 hours after the administration. Animals were euthanized with CO₂ 2 h after LPS injection. Cardiac blood was collected into EDTA-K2 vacutainers, and a part of the anticoagulated blood was centrifuged and the plasma was frozen at −80° C.

2. TNF-α Assay

Frozen plasma was thawed at room temperature and the concentration of TNF-α in the plasma was measured using ELISA kit.

3. Statistics

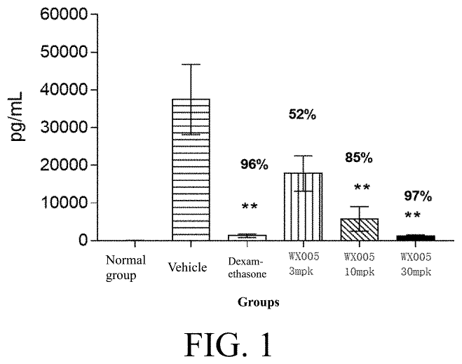

The experimental data were expressed using mean±SEM, and TNF-α levels were analyzed by One-way ANOVA. Significant differences were considered for p<0.05. The results of pharmacodynamic study evaluating LPS-induced TNF-α secretion in SD rats are shown in FIG. 1.

4. Results

FIG. 1 shows that: oral WX005 exhibited significant inhibitory effect on lipopolysaccharide (LPS)-induced TNF-α secretion. WX005 showed a clear dose-response relationship at doses from 3 mpk through 10 mpk to 30 mpk, while WX005 at 30 mpk in this experiment showed a potency equivalent to that of dexamethasone (DEX) at a dose of 0.5 mpk.

### Experimental Example 4: In Vivo Pharmacodynamic Study of WX005 in Human B-Cell Lymphoma OCI-LY10 Cells Subcutaneous Xenograft Tumor Mouse Model

1. Experimental Objective

The purpose of this experiment was to evaluate the efficacy of test drug WX005 on human B-cell lymphoma OCI-LY10 cell subcutaneous xenograft tumors in a CB17 SCID mouse model in vivo.

2. Experiment Materials

OCI-LY10 human B cell lymphoma cells, cultured in a 5% CO₂ incubator at 37° C. The culture medium comprised IMDM (GIBCO, Cat.# 12440053); supplemental components were 20% FBS (Hyclone, Cat.# SH30084.03) and 1% PenStrep (Thermo, Cat.# SV30010).

3. Experiment Procedures

OCI-LY10 tumor cells were subcultured. 0.2 mL (1×10⁷ cells) of OCI-LY10 cells (along with matrigel in a volume ratio of 1:1) was subcutaneously inoculated on the right back of each nude mouse, and the mice were administered in groups when the average tumor volume was 167 mm³. Animals were monitored daily for health and death, and routine examinations include observation of the effect of tumor growth and drug treatment on the daily performance of the animals, such as behavioral activities, food and water intake, weight changes (twice weekly), tumor size (twice weekly for tumor volume), appearance signs, or other abnormal conditions.

4. Data Analysis

The experimental indices were to investigate whether tumor growth was inhibited or delayed or the tumor was cured. The procedures comprised measuring tumor volume (TV), and calculating the tumor inhibiting therapeutic effect TGI (%) or relative tumor proliferation rate T/C (%) of the compound. TV=0.5a×b², where a and b represent the long diameter and short diameter of the tumor, respectively. TGI (%) =[(1−(average tumor volume at the end of administration in a treatment group−average tumor volume at the start of administration of the treatment group))/(average tumor volume at the end of treatment of the solvent control group−average tumor volume at the start of treatment of the solvent control group)]×100%.

T/C%=T_{RTV}/C_{RTV}×100% (T_{RTV}: RTV of treatment group; C_{RTV}: RTV of negative control group). Relative tumor volume (RTV) was calculated based on the results of tumor measurement. The calculation formula was: RTV=Vₜ/V₀, wherein Vo was the average tumor volume measured at the time of grouping and administration (i.e., d₀), Vₜ was the average tumor volume at a certain measurement, and the data of T_{RTV} and C_{RTV} were obtained on the same day.

5. Experiment Results

5.1. Mortality, morbidity and weight change

The body weight of the experimental animal was used as a reference index for indirectly measuring the toxicity of the medicament. No abnormality was observed in mice in the treatment groups after 18 days of treatment (PG-D1-D18), suggesting good tolerability.

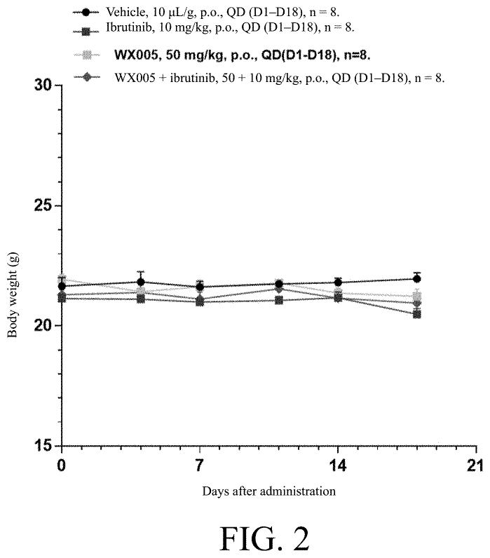

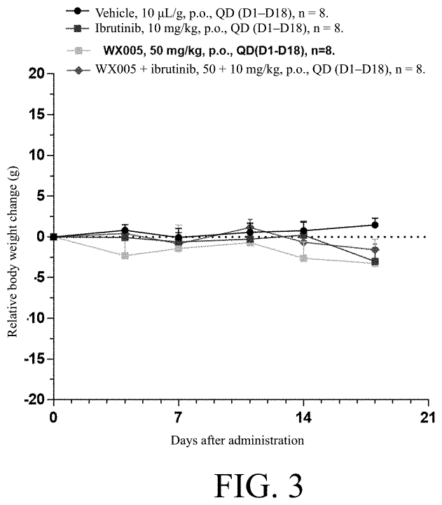

Effect of WX005 on body weight of human B-cell lymphoma OCI-LY10 cells subcutaneous xenograft tumor female CB17 SCID mouse model is shown in FIGS. 2 and 3. FIG. 2 illustrates the body weight change in different groups of human B-cell lymphoma OCI-LY10 cells subcutaneous xenograft tumor mouse models after WX005 was given. Data points represent the mean body weight of a group, and error bars represent standard error (SEM). The relative body weight changes shown in FIG. 3 were calculated based on the body weight of the animals at the beginning of administration. Data points represent the percentage changes of mean body weight of a group, and error bars represent standard error (SEM).

5.2. Tumor Growth Profile

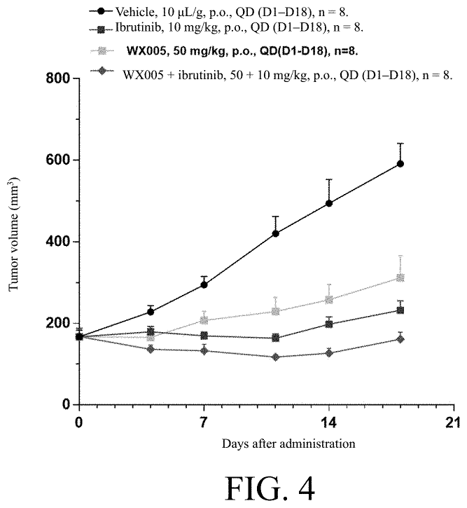

FIG. 4 illustrates the tumor growth profile in different groups of human B-cell lymphoma OCI-LY10 cells subcutaneous xenograft tumor mouse models after WX005 was given. Data points represent the mean tumor volume of a group, and error bars represent standard error (SEM).

6. Experiment Results and Discussion

In this experiment, we evaluated the in vivo efficacy of WX005 compound in a human B-cell lymphoma OCI-LY10 cells subcutaneous xenograft tumor model. Tumor volumes for each group at different time points are shown in FIG. 4.

At day 18 after the start of administration, the T/C value was 39%, the TGI value was 85%, and the p value was <0.001 in the ibrutinib (10 mpk) group. For the WX005 (50 mpk) group, the T/C value was 53%, the TGI value was 66%, and the p value was <0.01. For the WX005 +ibrutinib (50+10 mpk) group, the T/C value was 27%, the TGI value was 102%, and p was <0.001. The compound has a significant tumor inhibitory effect as compared to the solvent control group, and is significantly superior to that of the ibrutinib (10 mpk) group.

OCI-LY10 cell line is an ABC-DLBCL cell line highly dependent on MyD88-L265P and BCR (CD79A/B) double mutation. IRAK4 inhibitor WX005 (50 mpk) exhibited some tumor inhibitory effect as a monotherapy (TGI=66%) and good tolerability in animals; BTK inhibitor ibrutinib (10 mpk) also exhibited some tumor inhibitory effect (TGI=85%); when the WX005 (50 mpk) and the ibrutinib (10 mpk) were combined, the tumor inhibitory effect of ibrutinib (10 mpk) was significantly improved as compared with that of ibrutinib monotherapy, and the TGI reached 102%, suggesting the synergistic effect of the double inhibition of the BCR pathway and the MyD88 pathway, and a good tolerability in animals.

### Experimental Example 5: In Vivo Efficacy Assay of Collagen-Induced Mouse Arthritis 1. Experimental Objective

The purpose of this experiment was to investigate the therapeutic effect of compound WX005 in a mouse model of collagen-induced arthritis.

2. Experiment Materials

Animals: male DBA/1 mice aged 6-8 weeks; supplier: Vital River.

3. Experiment Reagents

LPS: Sigma; Cat. No.: L2630;

Acetic acid: Sigma (St. Louis, Mo., USA), Cat. No.: A8976;

Complete Freund's adjuvant: Sigma, Cat. No.: F5881;

Bovine collagen II: Sichuan University; Cat. No.: 20181016;

Vehicle: 5% DMSO+10% SOLUTOL+85% H₂O.

4. Experiment Instruments

Anesthesia machine: Raymain Information Technology, iR3TM HSIV-u

High-speed homogenator: IKA, T10 basic, 37140, 827825

5. Experiment Procedures

Grouping: Among 39 DBA/1 mice, 5 mice were randomly selected as normal controls, and the other 34 were immunized. The day of the first immunization was recorded as Day 0. During modeling, DBA/1 mice were anesthetized with isoflurane and sensitized by injecting 50 microliters of prepared collagen emulsion (containing 200 micrograms of CII) subcutaneously on the tail (2-3 cm from the root of the tail). On Day 23, 100 microliters of 0.3 mg/mL LPS solution (containing 30 micrograms of LPS) was intraperitoneally injected. Mice in the normal group were not immunized.

On Day 26, when the average clinical score reached about 0.8, 24 mice with clinical scores of 0-1 were selected, and the mice were randomly grouped into 3 treatment groups of 8 according to the body weight and the score.

The first group (normal group) was normal mice without treatment; vehicle was administered to the second group (vehicle group); the third group (WX005 group) was given WX005 at a dose of 100 mg/kg twice daily for 14 days. The volume for oral gavage was 10 mL/kg (Table 7).

TABLE 7
Grouping of experiment

| | Number of | Administered | Route of | Dose and |
|---|---|---|---|---|
| Grouping | animals | compounds | administration | frequency |
| Normal group | 5 | NA | NA | NA |
| Vehicle group | 8 | NA | Oral gavage | Once daily |
| WX005 group | 8 | WX005 | Oral gavage | 100 mpk, |
| | | | | twice daily |

Note:
NA represents no administration.

Clinical observation: the general health and body weight changes of DBA/1 mice were observed daily from 7 days before immunization to Day 23 after immunization (recorded once weekly). After Day 23, mice were observed daily for health, morbidity, and weight changes (recorded at least three times a week) until the end of assay. Scoring according to the degree of lesion (redness, joint deformity) was performed on a scale of 0-4 points, with a maximum score of 4 for each limb and 16 for each animal. The scoring criterion is shown in Table 8.

TABLE 8
Clinical scoring criterion for arthritis

| Score | Symptoms |
|---|---|
| 0 | No erythema and redness |
| 1 | Erythema or mild redness near the tarsal bones or at the |
| | ankle joints or metatarsal bones, and redness in 1 toe |
| 2 | Slight erythema and redness of the ankle and metatarsal |
| | bones, or redness and swelling of more than two toes |
| 3 | Moderate erythema and swelling in the ankle, wrist, and |
| | metatarsals |
| 4 | Severe redness and swelling in the ankle, wrist, |
| | metatarsals and toes |

6. Results and Discussion

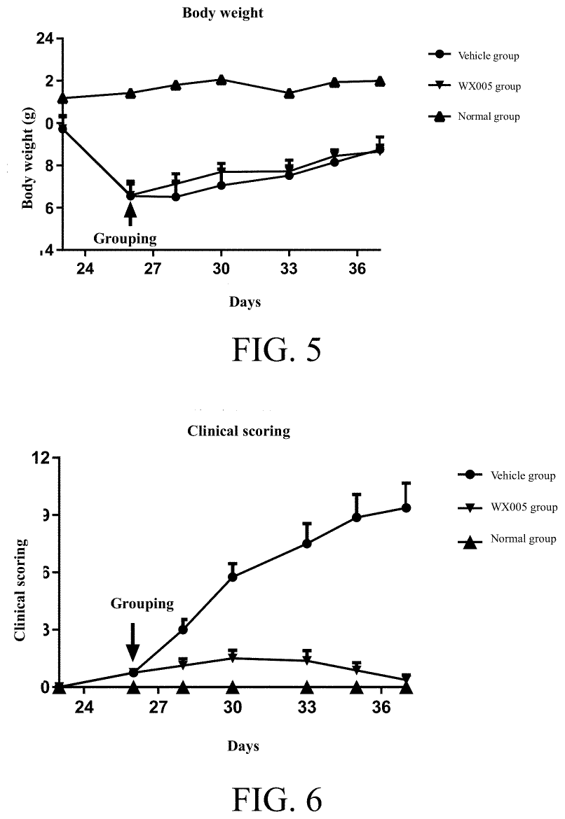

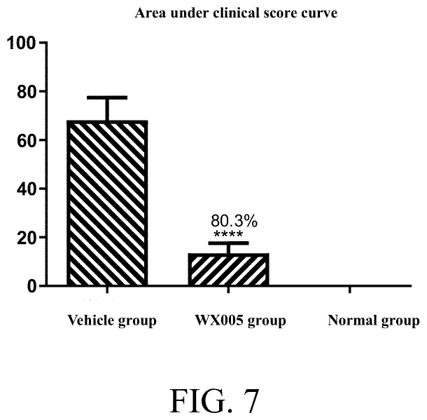

As shown in the data of FIG. 5, the body weight of the mice in the normal group did not increase significantly, and the body weight of the mice in the vehicle group and WX005 group increased steadily. As shown in FIGS. 6 and 7, the results of clinical scores of mice in vehicle group and WX005 group were summarized, and it can be seen that WX005 group exhibited excellent efficacy.

1. A compound of formula (II), an isomer thereof or a pharmaceutically acceptable salt thereof, 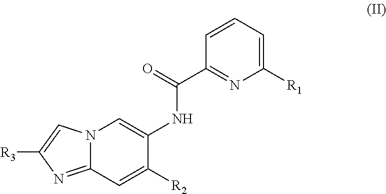
wherein,
R₁ is C₁₋₃ alkyl, wherein the C₁₋₃ alkyl is optionally substituted with 1, 2 or 3 Rₐ;
R₂ is selected from C₁₋₆ alkyl, C₁₋₆ alkoxy, cyclopropyl, azetidinyl, 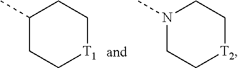
the C₁₋₆ alkyl, C₁₋₆ alkoxy, cyclopropyl, azetidinyl, 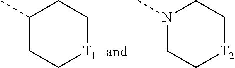
being optionally substituted with 1, 2 or 3 R_{b};
R₃ is C₁₋₆ alkyl, the C₁₋₆ alkyl being optionally substituted with 1, 2 or 3 R_{c};
T₁ is selected from CH₂, NH and O;
T₂ is selected from CH₂, NH and O;
each Rₐ is independently selected from H, F, Cl, Br, I, OH, NH₂, CN and CH₃;
each R_{b} is independently selected from H, F, Cl, Br, I, OH, NH₂, CN, CH₃, —C(═O)—C₁₋₃ alkyl, —C(=O)—C₁₋₃ alkoxy, —C(=O)NH₂ and —COOH, the CH₃, —C(═O)—C₁₋₃ alkyl and —C(═O)—C₁₋₃ alkoxy being optionally substituted with 1, 2 or 3 R;
each R_{c} is independently selected from H, F, Cl, Br, I, OH, NH₂, CN, CH₃, COOH and —S(═O)₂—C₁₋₃ alkyl; and
each R is independently selected from H, OH and NH₂.

2. The compound, the isomer thereof or the pharmaceutically acceptable salt thereof according to claim 1, wherein R₁ is CF₃.

3. The compound, the isomer thereof or the pharmaceutically acceptable salt thereof according to claim 1, wherein each R_{b} is independently selected from H, F, Cl, OH, NH₂, CN, CH₃, CH₂OH, CH₂NH₂, 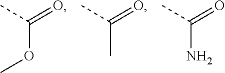
and —COOH.

4. The compound, the isomer thereof or the pharmaceutically acceptable salt thereof according to claim 1, wherein R₂ is selected from alkyl, C₁₋₃ alkoxy, 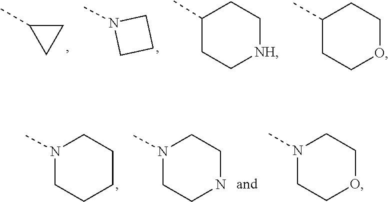
the C₁₋₃ alkyl, C₁₋₃ alkoxy, 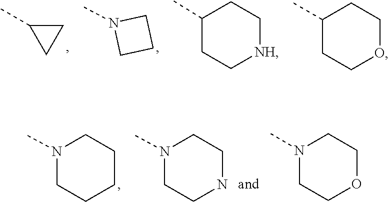
being optionally substituted with 1, 2, or 3 R_{b}.

5. The compound, the isomer thereof or the pharmaceutically acceptable salt thereof according to claim 3, wherein R₂ is selected from

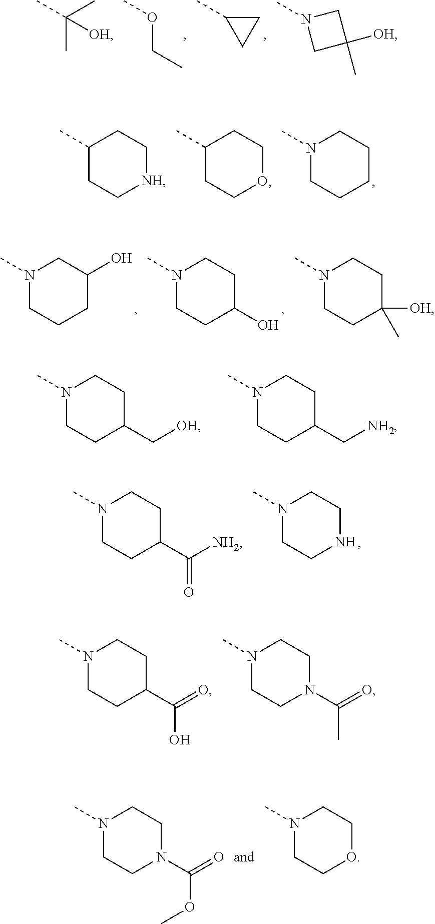

6. The compound, the isomer thereof or the pharmaceutically acceptable salt thereof according to claim 1, wherein each R_{c} is independently selected from H, F, Cl, OH, NH₂, COOH and —S(═O)₂CH₃.

7. The compound, the isomer thereof or the pharmaceutically acceptable salt thereof according to claim 1, wherein R₃ is selected from

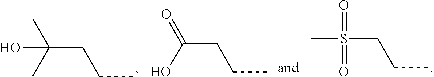

8. The compound, the isomer thereof, or the pharmaceutically acceptable salt thereof according to claim 1, wherein the compound, the isomer thereof, or the pharmaceutically acceptable salt thereof is selected from: 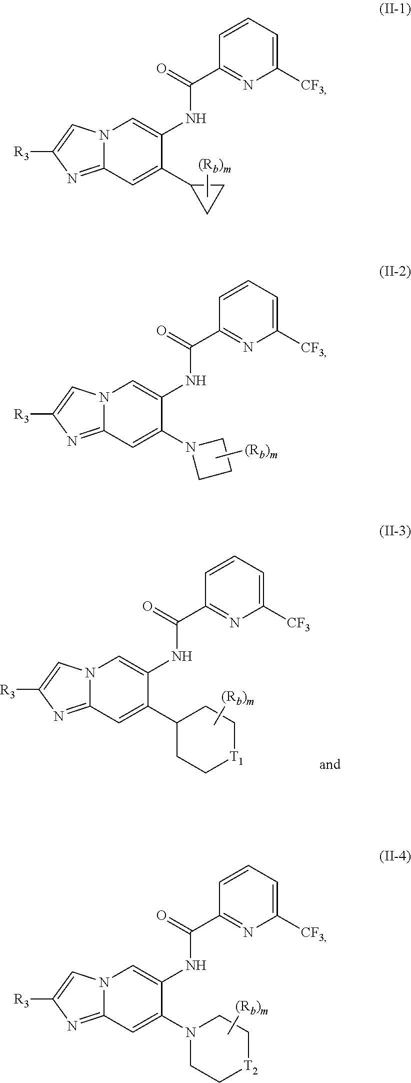
wherein R₃ is as defined in claim 1 or 7;
R_{b} is as defined in claims 1 or 3;
T₁ and T₂ are as defined in claim 1;
m is selected from 1, 2 and 3.

9. A compound of the following formulae, an isomer thereof or a pharmaceutically acceptable salt thereof:

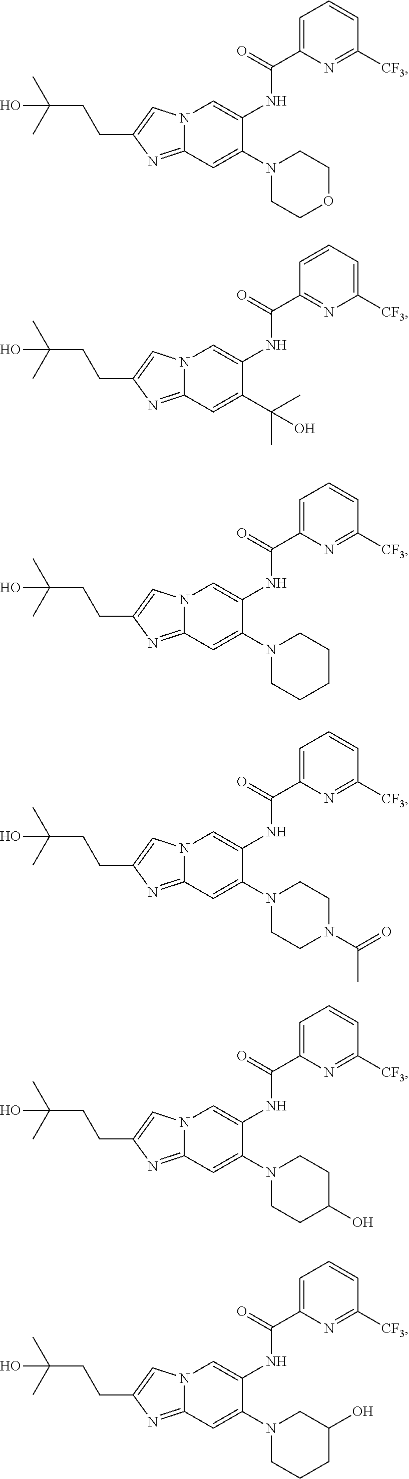

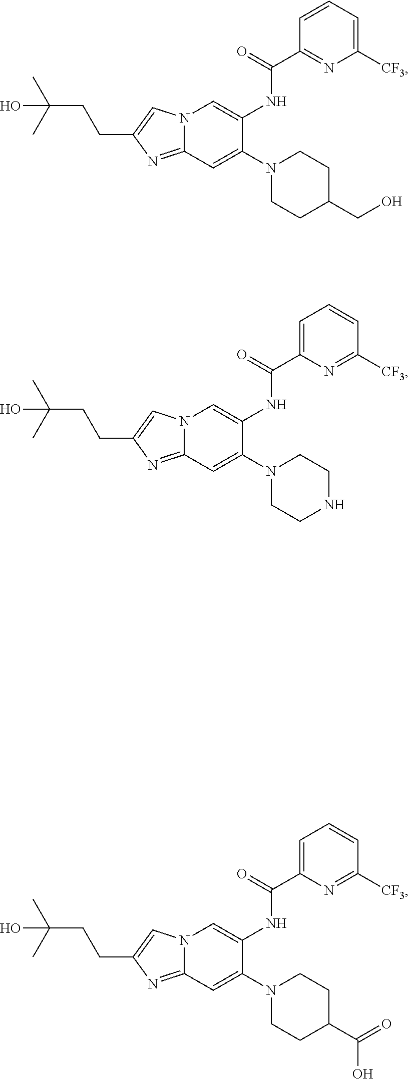 [SMILES: CC(C)(O)CCc1cn2cc(NC(=O)c3cccc(C(F)(F)F)n3)c(N3CCC(CO)CC3)cc2n1 ; CC(C)(O)CCc1cn2cc(NC(=O)c3cccc(C(F)(F)F)n3)c(N3CCNCC3)cc2n1 ; CC(C)(O)CCc1cn2cc(NC(=O)c3cccc(C(F)(F)F)n3)c(N3CCC(C(=O)O)CC3)cc2n1]

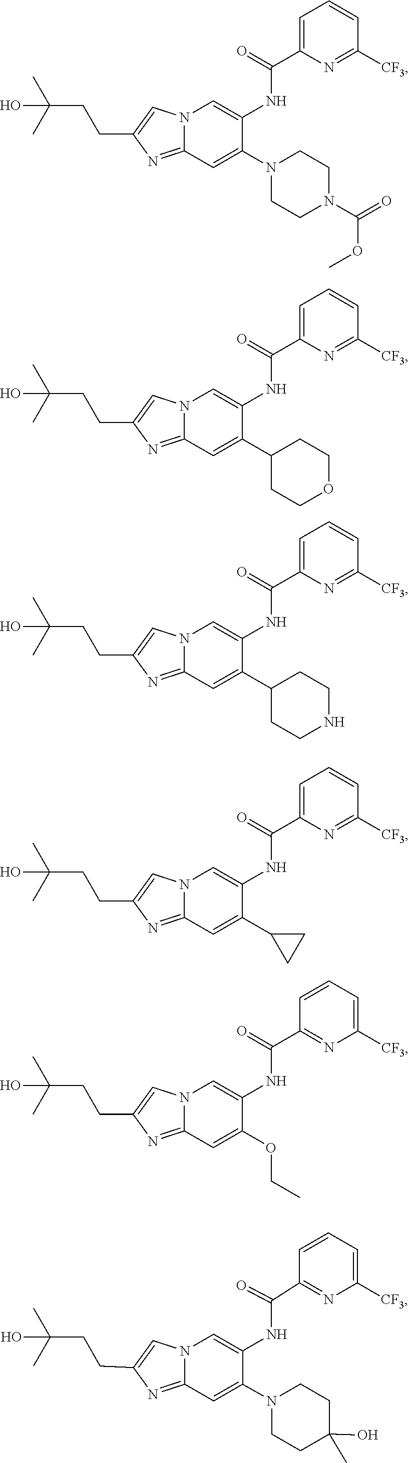

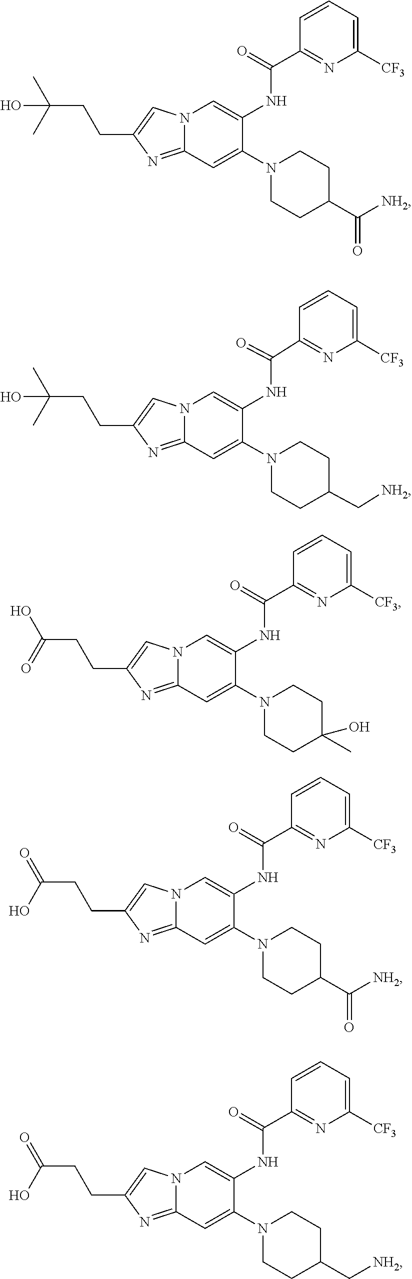

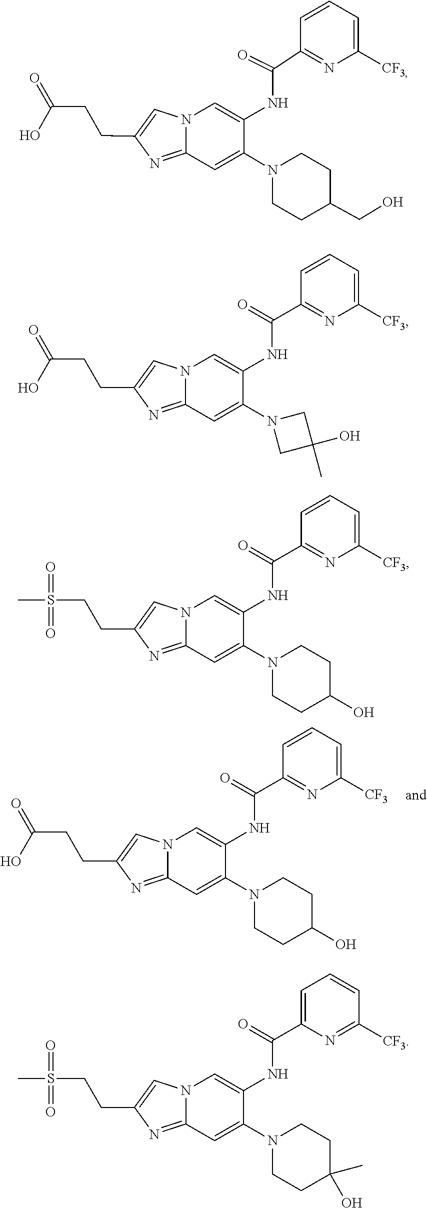

10. A pharmaceutical composition comprising a therapeutically effective amount of the compound, the isomer thereof or the pharmaceutically acceptable salt thereof according to claim 1 as an active ingredient, and a pharmaceutically acceptable carrier.

11. Use of the compound, the isomer thereof or the pharmaceutically acceptable salt thereof according to claim 1 in the preparation of a medicament for the treatment of an IRAK4-related disease.

12. Use of the composition according to claim 10 in the preparation of a medicament for the treatment of an IRAK4-related disease.
